SEQUENCE LISTING (4 sequences)

SEQ ID NO: 1 - DNA, 20 bp, Artificial Sequence
  Primer sequences
gaacggcatcaaggtgaact

SEQ ID NO: 2 - DNA, 20 bp, Artificial Sequence
  Primer Sequences
tgctcaggtagtggttgtcg

SEQ ID NO: 3 - DNA, 24 bp, Artificial Sequence
  Primer sequences
cggttccgatgccctgaggctctt

SEQ ID NO: 4 - DNA, 24 bp, Artificial Sequence
  Primer sequences
cgtcacacttcatgatggaattga

(19) United States
(12) Patent Application Publication
(10) Pub. No.: US 2022/0125856 A1
(43) Pub. Date: Apr. 28, 2022

(54) USE OF OVARIAN-DERIVED HYDROGELS FOR RESTORATION OF REPRODUCTIVE FUNCTION AND HEALTH IN WOMEN

(71) Applicants: UNIVERSITY OF PITTSBURGH - OF THE COMMONWEALTH SYSTEM OF HIGHER EDUCATION, Pittsburgh, PA (US); UPMC, Pittsburgh, PA (US)

(72) Inventors: Michael Joseph BUCKENMEYER, Middletown, MD (US); Kyle E. ORWIG, Wexford, PA (US); Bryan Nicklaus BROWN, Pittsburgh, PA (US); Meena SUKHWANI, Pittsburgh, PA (US)

(73) Assignees: UNIVERSITY OF PITTSBURGH - OF THE COMMONWEALTH SYSTEM OF HIGHER EDUCATION, Pittsburgh, PA (US); UPMC, Pittsburgh, PA (US)

(21) Appl. No.: 17/566,504

(22) Filed: Dec. 30, 2021

Related U.S. Application Data
(63) Continuation of application No. PCT/US2020/041858, filed on Jul. 13, 2020.
(60) Provisional application No. 62/873,263, filed on Jul. 12, 2019.

(51) Int. Cl. A61K 35/54; A61K 9/06; A61K 9/00; A61P 15/08

(52) U.S. Cl. CPC A61K 35/54; A61P 15/08; A61K 9/0034; A61K 9/06

(57) ABSTRACT

The present disclosure provides methods for restoring reproductive health and/or fertility in a subject by delivering immature follicles to the subject using an ovarian-derived hydrogel. The present disclosure also provides methods for treating ovarian-associated diseases using ovarian-derived hydrogels or their derivatives for delivery of therapeutic agents to the subject in need thereof. The present disclosure further provides kits for restoring reproductive health and/or fertility, and kits for treating ovarian-associated diseases.

### 1. CROSS-REFERENCE TO RELATED APPLICATION

This application is a continuation of International Patent Application No. PCT/US2020/041858, filed Jul. 13, 2020, which claims priority to U.S. Provisional Application Ser. No. 62/873,263 filed Jul. 12, 2019, the contents of which are hereby incorporated by reference in their entireties.

### 2. SEQUENCE LISTING

This application contains a Sequence Listing, which has been submitted in ASCII format via EFS-Web, and is hereby incorporated by reference in its entirety. The ASCII copy, created on Dec. 29, 2021, is named Sequence_Listing_072396.0897.TXT and is 1,070 bytes in size.

### 3. TECHNICAL FIELD

The present disclosure provides methods and kits for restoring reproductive health and/or fertility and for treating ovarian-associated diseases.

### 4. BACKGROUND

Damage or dysfunction of ovarian tissues and cells can lead to a myriad of health problems resulting in the onset and persistence of both local and systemic diseases, such as ovarian cancer, endometriosis, polycystic ovarian syndrome (PCOS), and premature ovarian failure (POF). Certain treatments for ovarian-associated diseases can have direct or indirect consequences on the ovarian microenvironment, which negatively affects the quality of life in female patients.

The number of female cancer survivors has risen significantly in recent years, and many patients experience infertility due to the detrimental effects of chemotherapy and radiation therapy. These cancer treatments may create irreversible damages in ovarian tissues and ovarian follicles. Ovarian follicles control oocyte development and contribute to overall endocrine function. Premature depletion of ovarian follicles is associated with several health problems, including infertility.

Certain limited options are available to restore fertility for reproductive-age women in remission. One clinical procedure is orthotopic transplantation of a patient's own healthy cryopreserved ovarian cortical strips. The strips contain immature primordial follicles and have the potential to restore normal ovarian function. However, this procedure does not necessarily guarantee the removal of residual malignant cells and may lead to a recurrence of cancer. In addition, this procedure can be invasive and associated with a risk of complications.

Certain methods for assisted reproductive technologies, such as in vitro follicle maturation (IVM), can have the benefit of eliminating the risk of reintroducing malignant cells. IVM involves the steps of isolating and screening follicles from ovarian tissues from the cancer patients to remove malignant cells. Moreover, before being transplanted into the patients, isolated follicles can be cultured in vitro for an extended period of time, to let the contents of follicles and oocytes inside mature in vitro. Such in vitro maturation process can involve contacting the follicles with hormone, growth factors, steroids, and other maturation factors using multi-step protocols. Certain IVM techniques present a challenge to use in humans because of the difficulties in developing and maturing human follicles in vitro.

Therefore, there remains a need for methods of restoring reproductive health and/or fertility in female subjects.

### 5. SUMMARY

The present disclosure provides methods for restoring reproductive health and/or fertility in a subject by delivery of immature follicles to the subject using an ovarian-derived hydrogel. The present disclosure also provides methods for treating ovarian-associated diseases using ovarian-derived hydrogels or their derivatives for delivery of therapeutic agents to the subject in need thereof. The present disclosure further provides kits for restoring reproductive health and/or fertility, and kits for treating ovarian-associated diseases.

In one aspect, the present disclosure provides methods for restoring reproductive health and/or fertility in a subject. An example method includes providing an immature follicle, mixing the immature follicle with a hydrogel to form a follicle-hydrogel mixture, and delivering the follicle-hydrogel mixture to an ovary of the subject, wherein the immature follicle matures in vivo in the ovary.

In certain embodiments, the subject has a cancer. In certain embodiments, the cancer is in remission. In certain embodiments, the subject has previously received a cancer treatment. In certain embodiments, the cancer treatment is a chemotherapy or a radiation therapy.

In certain embodiments, the immature follicle is isolated from an ovarian tissue of the subject. In certain embodiments, the subject has no detectable endogenous follicle or has few detectable endogenous follicles. In certain embodiments, the subject is infertile. In certain embodiments, the ovarian tissue is obtained from the subject before the subject receives the cancer treatment. In certain embodiments, the immature follicle is isolated by an enzymatic treatment of the ovarian tissue.

In certain embodiments, the mixing includes storing the follicle-hydrogel mixture under conditions that prevent gelation and preserve viability of the immature follicle. In certain embodiments, the mixing includes storing the follicle-hydrogel mixture at about 4° C.

In certain embodiments, the immature follicle is selected from a primordial follicle, a primary follicle, a secondary follicle, and combinations thereof.

In certain embodiments, delivering the follicle-hydrogel mixture to an ovary of the subject includes delivering the follicle-hydrogel mixture to the ovary of the subject by a microinjection needle. In certain embodiments, delivering the follicle-hydrogel mixture to an ovary of the subject includes delivering the follicle-hydrogel mixture to the ovary of the subject using a minimally invasive procedure. In certain embodiments, delivering the follicle-hydrogel mixture to an ovary of the subject includes delivering the follicle-hydrogel mixture to the ovary of the subject under the guidance of an ultrasound.

In certain embodiments, the hydrogel includes a decellularized ovarian tissue. In certain embodiments, decellularized ovarian tissue is from a mammal.

In certain embodiments, the hydrogel includes an extracellular matrix of the decellularized ovarian tissue, wherein the extracellular matrix is present in a concentration between about 1 mg/ml to about 10 mg/ml. In certain embodiments, the hydrogel comprises one or more ovary-specific components. The one or more ovary-specific component is selected from the group consisting of anti-mullerian hormone (AMH), estradiol, insulin growth factor, progesterone, and combinations thereof.

In another aspect, the present disclosure provides methods for treating an ovarian-associate disease. In exemplary embodiments, the method includes providing a therapeutic component, mixing the therapeutic component with an ovarian tissue derivative to form a therapeutic component-derivative mixture, and delivering the mixture to an ovary of a subject.

In certain embodiments, the therapeutic component is selected from biomaterials, stem cells, therapeutic agents, and combinations thereof. In certain embodiments, the ovarian tissue derivative includes a decellularized ovarian tissue.

In certain embodiments, the ovarian tissue derivative is selected from the ovarian tissue powder, ovarian tissue microparticles, ovarian tissue nanoparticles, ovarian tissue soluble fractions, ovarian tissue hydrogels, ovarian tissue matrix-bound nanovesicles, and combinations thereof.

In certain embodiments, the therapeutic component-derivative mixture includes an extracellular matrix of the decellularized ovarian tissue, wherein the extracellular matrix is present in a concentration between about 1 mg/ml to about 10 mg/ml. In certain embodiments, the therapeutic component-derivative mixture comprises one or more ovary-specific components. The one or more ovary-specific component is selected from the group consisting of anti-mullerian hormone (AMH), estradiol, insulin growth factor, progesterone, and combinations thereof.

In another aspect, the present disclosure further provides kits for restoring reproductive health and/or fertility in a subject, including an immature follicle and a hydrogel.

In certain example embodiments, the kit further includes instructions for mixing the immature follicle with the hydrogel to form a follicle-hydrogel mixture and delivering the follicle-hydrogel mixture to an ovary of the subject, where the immature follicle matures in vivo in the ovary.

In certain embodiments, the subject has a cancer. In certain embodiments, the cancer is in remission. In certain embodiments, the subject has previously received a cancer treatment. In certain embodiments, the cancer treatment is a chemotherapy or a radiation therapy.

In certain embodiments, the immature follicle is isolated from an ovarian tissue of the subject. In certain embodiments, the subject has no detectable endogenous follicle or has few detectable endogenous follicles. In certain embodiments, the subject is infertile. In certain embodiments, the ovarian tissue is obtained from the subject before the subject receives the cancer treatment. In certain embodiments, the immature follicle is isolated by an enzymatic treatment or a mechanical isolation of the ovarian tissue.

In certain embodiments, the mixing includes storing the follicle-hydrogel mixture under conditions that prevent gelation and preserve viability of the immature follicle. In certain embodiments, the mixing includes storing the follicle-hydrogel mixture at about 4° C.

In certain embodiments, the immature follicle is selected from the group consisting of a primordial follicle, a primary follicle, a secondary follicle, and combinations thereof.

In certain embodiments, delivering the follicle-hydrogel mixture to an ovary of the subject includes delivering the follicle-hydrogel mixture to the ovary of the subject by a microinjection needle. In certain embodiments delivering the follicle-hydrogel mixture to an ovary of the subject delivering the follicle-hydrogel mixture to the ovary of the subject using a minimally invasive procedure. In certain embodiments, delivering the follicle-hydrogel mixture to an ovary of the subject delivering the follicle-hydrogel mixture to the ovary of the subject under the guidance of an ultrasound.

In certain embodiments, the hydrogel includes a decellularized ovarian tissue. In certain embodiments, the decellularized ovarian tissue is from a mammal.

In another aspect, the present disclosure further provides kits for treating an ovarian-associate disease, including a therapeutic component and an ovarian tissue derivative.

In certain example embodiments, the kit includes instructions for mixing the therapeutic component with the ovarian tissue derivative to form a therapeutic component-derivative mixture; and delivering the mixture to an ovary of a subject.

In certain embodiments, the therapeutic component is selected from biomaterials, stem cells, therapeutic agents, and combinations thereof. In certain embodiments, the ovarian tissue derivative comprises a decellularized ovarian tissue.

In certain embodiments, the ovarian tissue derivative is selected ovarian tissue powder, ovarian tissue microparticles, ovarian tissue nanoparticles, ovarian tissue soluble fractions, ovarian tissue hydrogels, ovarian tissue matrix-bound nanovesicles, and combinations thereof.

The present disclosure also provides kits for restoring reproductive health and/or fertility in a subject. An example kit includes tools for obtaining an immature follicle and/or tools for delivering the immature follicle to a subject.

### 6. BRIEF DESCRIPTION OF THE DRAWINGS

FIG. 1A shows a graphical image of a non-limiting exemplary method for restoring fertility in accordance with the present disclosure.

FIGS. 1B-1D provide photographic images of (1B) a harvested ovary, (1C) a diced ovary, and (1D) a cubed ovary in a flask for decellularization in accordance with the present disclosure.

FIGS. 1E-1G provide photographic images of (1E) a decellularized ovarian tissues, (1F) frozen and lyophilized ovarian tissues, and (1G) powdered ovarian-specific extracellular matrix (OECM) in accordance with the present disclosure.

FIGS. 1H-1K provide native (top row) and decellularized (bottom row) images of (1H) DAPI, (1I) H&E, (1J) periodic acid-schiff (PAS), and (1K) scanning electron micrographs (SEM) in accordance with the present disclosure.

FIG. 1L shows a graph of PicoGreen assay in accordance with the present disclosure.

FIG. 1M shows a graph of hydroxyproline (HYP) concentrations in accordance with the present disclosure.

FIG. 1N shows a graph of sulfated-glycosaminoglycans (sGAG) levels in accordance with the present disclosure.

FIGS. 2A-2D provide immunohistochemistry (IHC) images of native (top row) and decellularized ovarian tissues (bottom row) illustrating expression of (2A) collagen I, (2B) collagen IV, (2C) laminin, and (2D) fibronectin in accordance with the present disclosure.

FIG. 2E shows a graph of an anti-mullerian hormone (AMH) level in accordance with the present disclosure.

FIG. 2F shows a graph of estradiol concentrations in accordance with the present disclosure.

FIG. 2G shows a graph of progesterone levels in accordance with the present disclosure.

FIG. 3A shows a photographic image of exemplary solubilized OECM in accordance with the present disclosure.

FIGS. 3B-3C provide SEM images of hydrogel architecture with (3B) 4 mg/ml and (3C) 8 mg/ml of OECM concentrations in accordance with the present disclosure.

FIGS. 3D-3H show graphs showing (3D) fiber diameter, (3E) fiber length, (3F) node density, (3G) pore size, and (3h) porosity of an exemplary OECM hydrogel in accordance with the present disclosure.

FIG. 3I-3K shows graphs of (3I) storage moduli, (3J) loss moduli, and (3K) turbidimetric gelation kinetics of an exemplary OECM hydrogel in accordance with the present disclosure.

FIGS. 4A-4E shows IHC images of ovarian tissues, where (4A) 12-100 mg/kg, (4B) 12-200 mg/kg, (4C) 24-100 mg/kg, (4D) 24-200 mg/kg, and (4E) 0 mg/kg of busulfan-cyclophosphamide were administered via single IP injection in accordance with the present disclosure.

FIG. 4F shows a graph of follicle number in accordance with the present disclosure.

FIG. 4G shows a graph of follicle reduction in accordance with the present disclosure.

FIG. 4H shows images of bright field (top) and fluorescence (bottom) of intact follicles in accordance with the present disclosure.

FIG. 4I shows bright-field images of (top) gross morphology of an ovary during microinjection and (bottom) injection site for follicle transplant in accordance with the present disclosure.

FIG. 4J shows (top) brightfield and (bottom) fluorescence images showing transplanted OECM hydrogel injected via pressurized microinjection in accordance with the present disclosure.

FIG. 4K shows a multiphoton image, where GFP follicles, OECM are co-localized within an ovarian cortex to form an in situ ovary in accordance with the present disclosure.

FIGS. 5A-5B show (5A) GFP/nu-001 and (5B) GFP/ni-002 pubs that were born in consecutive liters from the same mother as a direct result of follicle transplantation in accordance with the present disclosure.

FIG. 5C shows a GFP pub delivered from a ciPOF female mouse during the third mating cycle in accordance with the present disclosure.

FIG. 5D-5E show (5D) outbred (i.e., GFP/nu×DBA) and (5E) inbred (i.e., GFP/nu×GFP/nu) mice in accordance with the present disclosure.

FIG. 5F shows genotyping results of GFP pubs and littermate controls in accordance with the present disclosure.

FIG. 5G shows an exemplary image of GFP positive follicles within a transplanted ovary in accordance with the present disclosure.

FIG. 5H shows an exemplary image of endogenous secondary follicles in accordance with the present disclosure.

FIG. 5I-5L show images of various follicle development stages including (5I) primordial, (5J) secondary, (5K) antral, and (5L) corpus luteum in accordance with the present disclosure.

FIGS. 6A-6C shows an exemplary microinjection procedure disclosed herein. (6A) follicles and OECM hydrogel mixture were loaded into a glass needle. (6B) The needle was positioned perpendicular to the ovary. (6C) A small volume of the mixture was injected into the ovarian cortex.

FIG. 7 shows an exemplary agarose gel used to characterize the presence of DNA within ovarian tissues post-decellularization in accordance with the present disclosure.

FIGS. 8A-8D shows IHC images illustrating (8A) primordial follicles, (8B) primary follicles, (8C) secondary follicles, (8D) antral follicles in accordance with the present disclosure.

FIGS. 9A-9D shows merged DAPI and FITC images illustrating (9A) left ovary of non-injected control 1, (9B) right ovary ofnon-injected control 1, (9C) left ovary of non-injected control 2, (9D) right ovary of non-injected control 2 in accordance with the present disclosure.

FIG. 10A provides an exemplary bright field image of OECM droplets.

FIG. 10B provides an exemplary microscopic image of an OECM droplet. Arrow points to an encapsulated follicle.

### 7. DETAILED DESCRIPTION

The present disclosure provides methods for restoring reproductive health and/or fertility in a subject by delivery of immature follicles to the subject using an ovarian-derived hydrogel. The present disclosure also provides methods for treating ovarian-associated diseases using ovarian-derived hydrogels or their derivatives for delivery of therapeutic agents to the subject in need thereof.

Unlike certain procedures in restoring fertility, the presently disclosed methods carry little risk in transplanting malignant cells back to the patients. The presently disclosed methods also eliminate any need for maturing follicles and oocytes in vitro. Thus, the presently disclosed subject matter offer a natural alternative for patients who wish to start a family.

Non-limiting embodiments of the disclosed subject matter are described by the present specification and Examples.

For purposes of clarity of disclosure and not by way of limitation, the detailed description is divided into the following subsections:

7.1 Definitions;

7.2 Methods for restoring reproductive health and/or fertility;

7.3 Methods for treating ovarian-associated diseases;

7.4 Ovarian-derived hydrogels; and

7.5 Kits.

### 7.1 Definitions

The terms used in this specification generally have their ordinary meanings in the art, within the context of this disclosed subject matter and in the specific context where each term is used. Certain terms are discussed below, or elsewhere in the specification, to provide additional guidance to the practitioner in describing the compositions and methods of the disclosed subject matter and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” of a substance as that term is used herein is that amount sufficient to effect beneficial or desired results, including clinical results, and, as such, an “effective amount” depends upon the context in which it is being applied. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

7.2 Methods for Restoring Reproductive Health and/or Fertility

The present disclosure provides methods for restoring reproductive health and/or fertility in a subject. The presently disclosed methods include providing an immature follicle, mixing the immature follicle with a hydrogel to form a follicle-hydrogel mixture, and delivering the follicle-hydrogel mixture to an ovary of the subject, where the immature follicle matures in vivo in the ovary of the subject.

In certain embodiments, the subject has no detectable endogenous follicles. In certain embodiments, the subject has few detectable endogenous follicles. In certain embodiments, the subject is infertile.

In certain embodiments, the subject has a cancer. In certain embodiments, the subject is a human subject. Non-limiting exemplary cancers that the subject has include adenoid cystic carcinoma, adrenal gland cancer, amyloidosis anal cancer, ataxia-telangiectasia, atypical mole syndrome, basal cell carcinoma, bile duct cancer, Birt Hogg Dube syndrome, bladder cancer, bone cancer, brain tumor, breast cancer, carcinoid tumor, cervical cancer, colorectal cancer, ductal carcinoma, endometrial cancer, esophageal cancer, gastric cancer, gastrointestinal stromal tumor—GIST, HER2-positive breast cancer, islet cell tumor, juvenile polyposis syndrome, kidney cancer, laryngeal cancer, liver cancer, lobular carcinoma, lung cancer, lymphoma, malignant glioma, melanoma, meningioma, multiple myeloma myelodysplastic syndrome (MDS), nasopharyngeal cancer, neuroendocrine tumor, oral cancer, osteosarcoma ovarian cancer, pancreatic cancer, pancreatic neuroendocrine tumors, parathyroid cancer, penile cancer, peritoneal cancer, Peutz-Jeghers syndrome, pituitary gland tumor, polycythemia vera, prostate cancer, renal cell carcinoma, retinoblastoma, salivary gland cancer, sarcoma, skin cancer, small intestine cancer, stomach cancer, testicular cancer, thymoma thyroid cancer, uterine (endometrial) cancer, vaginal cancer, and Wilms' tumor.

In certain embodiments, the subject is receiving a cancer treatment. In certain embodiments, the subject has previously received a cancer treatment. In certain embodiments, the cancer is in remission. In certain embodiments, the subject is not receiving a cancer treatment due to the cancer remission.

In certain embodiments, the cancer treatment is a chemotherapy or a radiation therapy. In certain embodiments, the cancer treatment has caused damages in ovarian tissue of the subject, resulting in a dysfunction of the ovarian tissue. In certain embodiments, the subject is infertile.

In certain embodiments, the immature follicle is obtained from a donor subject that is different from the subject receiving the immature follicle. In certain embodiments, the immature follicle is allogeneic immature follicle. In certain embodiments, the donor subject has a healthy reproductive function.

In certain embodiments, the immature follicle is obtained from the subject receiving the immature follicle. In certain embodiments, the immature follicle is autologous immature follicle. In certain embodiments, the immature follicle is obtained from the subject before the subject receives the cancer treatment. As such, no damage associated with the cancer treatment has occur in the ovary of the subject.

In certain embodiments, the immature follicles are isolated from an ovarian tissue obtained from the subject. In certain embodiments, the immature follicles are isolated from ovarian cortex of the ovarian tissue. Any methods known in the art for isolation of follicles from an ovarian tissue can be used with the presently disclosed subject matter. Non-limiting exemplary methods for isolation of follicles from an ovarian tissue are disclosed in Kim et al., NPJ Regen Med. 2016; 1: 16010; Vanacker et al., Fertil Steril. 2011 August; 96(2):379-383; Kim et al., Reprod Sci. 2018 August; 25(8):1270-1278; Mouloungui et al, J Ovarian Res. 2018 Jan. 5; 11(1):4; Chiti et al., J Ovarian Res. 2017 Oct. 23; 10(1):71; Follicle Handbook, Woodruff and Shea Labs, November 2014, accessible at https://www.woodrufflab.org/collaborations. To ensure the viability of the follicles to be injected, isolated immature follicles should be incubated in vitro for a short period of time before injection. Viability of follicles may decrease with prolonged incubation ex vivo. In certain embodiments, the isolated immature follicle may be stored at about 4° C. for up to about 30 minutes, up to about 1 hour, up to about 2 hours, up to about 3 hours, or up to about 4 hours.

In certain embodiments, the immature follicles are isolated from the ovarian tissue through enzymatic treatment of the ovarian tissue.

In certain embodiments, the immature follicles are isolated from the ovarian tissue using a mechanical isolation approach as disclosed in Follicle Handbook, Woodruff and Shea Labs, November 2014, accessible at https://www.woodrufflab.org/collaborations, the contents of which are incorporated herein by reference in its entirety.

In certain embodiments, the immature follicles are selected from primordial follicles, early primary follicles, primary follicles, secondary follicles, Graafian follicles, and combinations thereof. In certain embodiments, the immature follicles are selected from primordial follicles, primary follicles, secondary follicles, and combinations thereof. In certain embodiments, upon enzymatic treatment of the ovarian tissue, immature follicles are selected and isolated from the ovarian tissue. Any methods known in the art can be used for selecting immature follicles. Non-limiting exemplary methods for selecting immature follicles include sedimentation (e.g., Kim et al., NPJ Regen Med. 2016; 1: 16010), flow cytometry, and microdissection.

In certain embodiments, the isolated and selected immature follicles are free of malignant cells.

In certain embodiments, the presently disclosed methods further include mixing the immature follicle with a hydrogel to form a follicle-hydrogel mixture. In certain embodiments, the hydrogel is an ovarian-derived hydrogel. In certain embodiments, the hydrogel includes decellularized ovarian tissue. In certain embodiments, the methods disclosed herein include mixing the immature follicle with a hydrogel, where the hydrogel is a pre-gel solution. In certain embodiments, gelation has not occurred in the pre-gel solution. See Section 5.4 for hydrogels that can be used with the present disclosure.

In certain embodiments, the methods disclosed herein further include storing the follicle-hydrogel mixture under conditions that prevent gelation and preserve viability of the immature follicle, before delivery of the mixture to the subject. In certain embodiments, the follicle-hydrogel mixture is stored at about 4° C. In certain embodiments, the follicle-hydrogel mixture is stored on ice.

In certain embodiments, the methods disclosed herein include delivering the follicle-hydrogel mixture to the ovarian of the subject, where the immature follicle mature in vivo. In certain embodiments, the immature follicle matures in vivo in the ovary of the subject. As such, the presently disclosed methods eliminate the need for maturation of follicles and oocytes in vitro. Any methods known the art for delivering agents, materials, or cells to internal organs of a subject can be used with the presently disclosed subject matter. In certain embodiments, the follicle-hydrogel mixture is delivered to the ovary through a needle. In certain embodiments, the needle is a microinjection needle. In certain embodiments, the methods disclosed herein include introducing a needle into the ovary of the subject. In certain embodiments, the methods disclosed herein include introducing a needle into the ovarian cortex of the subject.

Minimally invasive procedures, such as transvaginal oocyte retrieval (TVOR) and amniocentesis are performed under the guidance of ultrasound, and have the benefit of operating with less damage to the body than with open surgery. Additionally, minimally invasive procedures are associated with less pain, a shorter hospital stay and fewer complications than open surgeries. In certain embodiments, the presently disclosed methods are performed using a minimally invasive procedure. In certain embodiments, the minimally invasive procedure is performed under the guidance of ultrasound. In certain embodiments, the minimally invasive procedure disclosed herein is adapted from TVOR and amniocentesis procedures.

The overall volume of the follicle-hydrogel mixture delivered to the subject varies depending on the size of the ovary of the subject. In certain embodiments, the methods disclosed herein include delivering at least about 2 μL, at least about 5 μL, at least about 10 μL, at least about at least about 50 μL, at least about 100 μL, at least about 200 μL, at least about 300 μL, at least about 400 μL, at least about 500 μL of the follicle-hydrogel mixture to the ovary of the subject. In certain embodiments, the methods disclosed herein include delivering at most about 500 μL, at most about 600 μL, at most about 700 μL, at most about 800 μL, at most about 900 μL, at most about 1 mL of the follicle-hydrogel mixture to the ovary of the subject. In certain embodiments, the methods disclosed herein include delivering between about 2 μL and about 10 μL, between about 10 μL and about 1 ml, between about 10 μL and about 100 μL, between about 100 μL and about 500 μL, between about 500 μL and about 1 mL, between about 300 μL and about 800 μL of the follicle-hydrogel mixture to the ovary of the subject. In certain embodiments, the methods disclosed herein include delivering about 2 μL, about 5 μL, about 10 μL, about 50 μL, about 100 μL, about 200 μL, about 300 μL, about 400 μL, about 500 μL, about 600 μL, about 700 μL, about 800 μL, about 900 μL, or about 1 mL of the follicle-hydrogel mixture to the ovary of the subject. In certain embodiments, the subject is a human subject or a mouse.

In certain embodiments, the methods disclosed herein include delivering between about 2 μL and about 10 μL, or about 6 μL, of the follicle-hydrogel mixture to the ovary of the subject, wherein the subject is a mouse. In certain embodiments, the methods disclosed herein include delivering between about 10 μL and about 1 mL of the follicle-hydrogel mixture to the ovary of the subject, wherein the subject is a human.

In certain embodiments, the volume of the follicle-hydrogel mixture delivered to the subject can vary based on the target tissue volume. For example, from about 5% to about 50%, from about 5% to about 10%, from about 10% to about 20%, from about 20% to about 30%, from about 30% to about 40%, from about 40% to about 50%, from about 5% to about 20%, from about 20% to about 40%, from about 25% to about 50%, or from about 10% to about 30% of the size (e.g., volume) of a subject's ovary is injected.

Upon delivery to the ovary, the follicle-hydrogel mixture is exposed to physiological conditions in the ovary, and starts to form a gel. The gelation process allows the immature follicle to retain within the ovary, and mature in vivo naturally.

### 7.3 Methods for Treating Ovarian-Associated Diseases

The present disclosure further provides methods for treating an ovarian-associate disease. The methods disclosed herein include providing a therapeutic component, mixing the therapeutic component with an ovarian tissue derivative to form a therapeutic component-derivative mixture, and delivering the mixture to an ovary of a subject.

Non-limiting exemplary ovarian-associated disease includes endometriosis, ovarian cysts, ovarian epithelial cancer, ovarian germ cell tumors, ovarian low malignant potential tumors, polycystic ovarian syndrome (PCOS), and premature ovarian failure (POF).

In certain embodiments, the ovarian tissue derivative includes a decellularized ovarian tissue. In certain embodiments, the decellularized ovarian tissue is derived from a mammal. In certain embodiments, the ovarian tissue derivative is selected from the ovarian tissue powder, ovarian tissue droplets, ovarian tissue microparticles, ovarian tissue nanoparticles, ovarian tissue soluble fractions, ovarian tissue hydrogels, ovarian tissue matrix-bound nanovesicles, and combinations thereof.

In certain embodiments, the ovarian tissue derivates are produced using methods that are adapted from protocols known in the art for producing extracellular matrix (ECM) derivatives, for example, protocols disclosed in Hoganson et al., J Biomed Mater Res A. 2016 July; 104(7):1728-35; Link et al., J Tissue Eng Regen Med. 2018 December; 12(12):2331-2336; International Publication No. WO2017/024193; and Naba et al., J Vis Exp. 2015 Jul. 23; (101):e53057, the contents of which are incorporated by reference herein in their entireties.

Non-limiting exemplary therapeutic components are biomaterials, stem cells, therapeutic agents, and combinations thereof.

Any suitable therapeutic agents known in the art for treating or benefiting ovarian-associated diseases can be used with the presently disclosed methods. Non-limiting exemplary therapeutic agents include hormones, growth factors, antimicrobial agents, emollients, retinoids, topical steroids, and combinations thereof. In certain embodiment, the therapeutic agent is an ovarian specific hormone or an ovarian specific growth factor, including but not limited to, anti-Müllerian hormone (AMH), progesterone, estradiol, insulin growth factor (IGF-1), and combinations thereof. In certain embodiment, the therapeutic agent is a non-ovarian specific growth factor or a non-ovarian specific hormone, including but not limited to, basic fibroblast growth factor (bFGF), vascular endothelial growth factor (VEGF), transforming growth factors (TGF-α and TGF-β), acidic fibroblast growth factor (aFGF), hepatocyte growth factor (HGF), insulin-like growth factors 1 and 2 (IGF-I and IGF-2), platelet derived growth factor (PDGF), stromal derived factor I alpha (SDF-I alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, interferons, and combinations thereof.

In certain embodiments, the therapeutic agent is an antimicrobial agent. Non-limiting exemplary antimicrobial agents that can be used with the presently disclosed methods include isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide periodate, and combinations thereof.

In certain embodiments, the therapeutic agent is an anti-inflammatory agent. Non-limiting exemplary anti-inflammatory agents that can be used with the presently disclosed methods include NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin, and combinations thereof.

Another variation may include polymeric components or additional biologic components in addition to the ovarian-derived derivatives. Another variation can include the ovarian-derived derivative which has been seeded with cells prior to or at the time of injection. The integrated cells may remain after the ovarian-derived derivative has fully disintegrated. The integrated cells may also be cells that act as precursors to the final tissue that is formed when the ovarian-derived derivative has fully degraded. Cells may be autologous (obtained from the intended recipient), from an allogeneic or xenogeneic source or from any useful cell line, including, but not limited to, stem cells or precursor cells (cells that can differentiate into another cell type) that are capable of cellular growth, remodeling, and/or differentiation. Non-limiting exemplary cells that can be used with the presently disclosed methods include stem cells, precursor cells, mesothelial cells, fibroblast cells, epithelial cells, and combinations thereof. Various commercially available cell lines include Clonetics® Primary Cell Systems (Lonza Group, Inc., Switzerland), and ATCC.

### 7.4 Ovarian-Derived Hydrogels

Any suitable hydrogels known in the art can be used with the presently disclosed subject matter. In certain embodiments, hydrogels disclosed in U.S. Patent Application No. 2017/0252485 can be used with the presently disclosed methods, and the contents of U.S. Patent Application No. 2017/0252485 are incorporated herein by reference in their entireties.

In certain embodiments, hydrogels used with the presently disclosed methods include decellularized ovarian tissue. In certain embodiments, the hydrogels are obtained through decellularization of an ovarian tissue (e.g., a mammalian ovarian tissue). The decellularized ovarian tissue retains ovarian specific components such as extracellular matrix (ECM) proteins, hormones, and growth factors. Thus, the hydrogel reorganizes site-specific ECM proteins and growth factors to form a porous scaffold (i.e., a matrix) that mimics the native ovarian microenvironment.

In certain embodiments, the disclosed hydrogels can comprise an extracellular matrix (ECM) of the decelluarized tissue. For example, the concentration of ovarian-derived ECM (i.e., the decellularized tissue) in the hydrogels can be from about 1 mg/ml to about 10 mg/ml. In certain embodiments, the concentration of ovarian-derived ECM in the ovarian-derived hydrogels can be from about 1 mg/mg to about 9 mg/ml, from about 1 mg/ml to about 8 mg/ml, from about 1 mg/ml to about 8 mg/ml, from about 1 mg/ml to about 7 mg/ml, from about 1 mg/ml to about 6 mg/ml, from about 1 mg/ml to about 5 mg/ml, from about 1 mg/ml to about 4 mg/ml, from about 1 mg/ml to about 3 mg/ml, from about 1 mg/ml to about 2 mg/ml, from about, or from about 4 mg/ml to about 8 mg/ml.

In certain embodiments, the disclosed hydrogel can comprise one or more ovary-specific components. The ovary specific component can include anti-mullerian hormone (AMH), estradiol, insulin growth factor, progesterone, or combinations thereof. For example, the presently disclosed hydrogel comprises at least one of the following ovarian hormones: at least about 2,000 pg/mL—AMH, at least about 150 pg/mL—estradiol, at least about 330 pg/mL—IGF-1, and/or at least about 25 ng/mL—progesterone. In certain non-limiting embodiments, the presently disclosed hydrogel comprises at from about 10% to about 50%, from about 15% to about 45%, from about 20% to about 40%, from about 21% to about 39%, from about 22% to about 38%, from about 23% to about 37%, from about 24% to about 36%, from about 25% to about 34%, from about 26% to about 33%, from about 27% to about 34%, from about 28% to about 33%, from about 29% to about 32%, or from about 30% to about 31% of AMH as compared to the native samples. In certain non-limiting embodiments, the presently disclosed hydrogel comprises at from about 100% to about 300%, from about 110% to about 290%, from about 120% to about 280%, from about 130% to about 270%, from about 140% to about 260%, from about 150% to about 250%, from about 160% to about 240%, from about 170% to about 230%, from about 180% to about 220%, or from about 190% to about 210% of estradiol as compared to the native samples. In certain non-limiting embodiments, the presently disclosed hydrogel comprises at from about 50% to about 200%, from about 60% to about 190%, from about 70% to about 180%, from 80% to about 170%, from about 90% to about 160%, from about 80% to about 150%, from about 90% to about 140%, from about 100% to about 130%, from about 105% to about 125%, or from about 110% to about 120% of IGF-1 as compared to the native samples. In certain non-limiting embodiments, the presently disclosed hydrogel comprises at from about 60% to about 100%, from about 65% to about 95%, from about 70% to about 90%, from about 72% to about 88%, from about 74% to about 86%, from about 76% to about 84%, or from about 78% to about 82% of progesterone as compared to the native samples. In certain non-limiting embodiments, the presently disclosed hydrogel comprises at least about 33% AMH, at least about 201% estradiol, at least about 124% IGF-1, at least about 81% progesterone as compared to the native samples.

In certain embodiments, the ovarian tissue is obtained from an autologous or a non-autologous source (relative to cells intended for delivery by the hydrogel). In certain non-limiting embodiments, the ovarian tissue is obtained from a non-autologous mammal, such as a syngeneic, allogeneic or xenogeneic mammal which may be of the same or a different species, such as a human or a non-human animal such as a non-human primate, a dog, a cat, a horse, a cow, a sheep, a goat, or a pig. The mammal may be of any age or at any stage of reproduction cycle. In certain embodiments, the ovarian tissue is obtained from a pig.

In certain embodiments, the hydrogel is produced by a method including decellularizing an ovarian tissue. In certain embodiments, decellularization of an ovarian tissue reduces genetic and immunogenic cellular components while retaining other ovary-specific components. In certain embodiments, the method further includes lyophilizing the decellularized ovarian tissue. In certain embodiments, the method further includes grinding (pulverizing, or otherwise rendering into smaller pieces) the lyophilized decellularized ovarian tissue. In certain embodiments, the method further includes digesting the lyophilized decellularized ovarian tissue. In certain embodiments, the method further includes solubilizing the digested decellularized ovarian tissue. In certain embodiments, the method further includes warming the solubilized decellularized ovarian tissue, where the warming allows physical crosslinking to occur. In certain embodiments, digesting the lyophilized decellularized ovarian tissue includes exposing the lyophilized decellularized ovarian tissue to fragmenting conditions, such as exposing the lyophilized decellularized ovarian tissue to pepsin and hydrochloride. In certain embodiments, solubilizing the digested decellularized ovarian tissue includes solubilizing the digested decellularized ovarian tissue in phosphate-buffered saline. In certain embodiments, warming the solubilized decellularized ovarian tissue includes warming the solubilized decellularized ovarian tissue to at least about 37° C. and maintaining the temperature below about 40° C.

In certain embodiments, the hydrogels may be prepared as follows. Ovarian tissues are decellularized using a minimal number of reagents and mild detergents to remove genetic material, while preserving ovary-specific components, such as growth factors, hormones, and ECM proteins. The decellularized ovarian tissue is lyophilized and ground into fine particles for digestion. The milled tissue is exposed to fragmenting conditions, e.g., the milled tissue is digested by pairing pepsin, a naturally occurring enzyme in the gastric submucosa, with hydrochloric acid (HCl) to form a viscous ECM digest. The ovarian hydrogels are formed by the addition of sodium hydroxide (NaOH) and phosphate-buffered saline to balance the pH and salt concentrations respectively. The collagen-rich components of the hydrogel form physical crosslinks that occur naturally at, for example, 37° C., which is ideal for in vivo applications.

In certain embodiments, ovarian tissue may be harvested and washed (e.g., with water or buffer) to remove excess blood present on the sample and extraneous tissue still connected to the ovaries. Ovaries can be frozen at −80° C. promptly after collection. If frozen, prior to decellularization, ovaries are thawed in ice-cold 1× phosphate-buffered saline (PBS) then diced into small cubes (sample volume of about 0.125 cm³ which equals to sample dimension of about 0.5 cm) and transferred to a flask with fresh 1× PBS. Diced tissues are stored overnight in 1×PBS at 4° C. Decellularization may then be performed.

In certain embodiments, the decellularization process can include a series of still or agitated washes (e.g., 300 rpm): water (Type 1), 0.02% trypsin/0.05% EDTA (1 Hr at 37° C.), 3.0% Triton X-100 (1 Hr), water rinse (Type 1, repeated), 4.0% sodium deoxycholate (1 Hr), 0.1% peracetic acid/4% ethanol (2 Hr), 1× PBS (15 min), water (15 min), water (15 min), 1× PBS (15 min). Following treatment samples are frozen at −80° C. then lyophilized.

In certain embodiments, enzymatic digestion product and hydrogel may be prepared as follows. Lyophilized scaffold materials are powdered (e.g., using a mill with a size 60 mesh screen). The powdered material is solubilized at a concentration of 20 mg/mL in a solution containing 1.0 mg/mL pepsin in 0.01N HCl at a constant stir rate of 300 rpm for 48 Hr. The digest solution is then be frozen at −80° C. until use. Enzymatic digestion is stopped by neutralizing the pH of the solution to 7.0 using 0.1N NaOH and diluting the solution to the desired concentration with 10× and 1× PBS. Gelation of the ovarian digest is induced by increasing the temperature of the gel into the physiologic range, e.g., about 37° C. to about 40° C.

In certain embodiments, the volume of the disclosed hydrogel can vary based on the target tissue volume. For example, from about 5% to about 50%, from about 5% to about 10%, from about 10% to about 20%, from about 20% to about 30%, from about 30% to about 40%, from about 40% to about 50%, from about 5% to about 20%, from about 20% to about 40%, from about 25% to about 50%, or from about 10% to about 30% of the size (e.g., volume) of a subject's ovary is injected.

In certain embodiments, the mechanical properties of the hydrogel can be modified through the addition of biocompatible crosslinking reagents such as, but not limited to, lysyl oxidase, genipin, ribose, rose bengal, or combinations thereof.

### 7.5 Kits

The present disclosure provides kits for restoring reproductive health and/or fertility in a subject. In certain embodiments, the kits include an immature follicle and a hydrogel. In certain embodiments, the kits include tools for obtaining immature follicles and/or tools for preparing and delivering the follicle-hydrogel mixture to the subject.

In certain embodiments, the immature follicle is obtained from a donor subject that is different from the subject receiving the immature follicle. In certain embodiments, the immature follicle is allogeneic immature follicle. In certain embodiments, the donor subject has a healthy reproductive function. In certain embodiments, the immature follicle is obtained from the subject receiving the immature follicle. In certain embodiments, the immature follicle is autologous immature follicle. In certain embodiments, the immature follicle is obtained from the subject before the subject receives the cancer treatment. As such, no damage associated with the cancer treatment has occur in the ovary of the subject.

In certain embodiments, the immature follicles are isolated from an ovarian tissue obtained from the subject. In certain embodiments, the immature follicles are isolated from ovarian cortex of the ovarian tissue. In certain embodiments, the immature follicles are selected from primordial follicles, early primary follicles, primary follicles, secondary follicles, Graafian follicles, and combinations thereof. In certain embodiments, the immature follicles are selected from primordial follicles, primary follicles, secondary follicles, and combinations thereof. In certain embodiments, upon enzymatic digestion of the ovarian tissue, immature follicles are selected and isolated from the ovarian tissue. In certain embodiments, the isolated and selected immature follicles are free of malignant cells.

In certain embodiments, the hydrogel is an ovarian-derived hydrogel. In certain embodiments, the hydrogel includes decellularized ovarian tissue. In certain embodiments, the methods disclosed herein include mixing the immature follicle with a hydrogel, where the hydrogel is a pre-gel solution, and gelation has not occurred in the pre-gel solution. See, supra, Section 5.4 for hydrogels that can be used with the present disclosure.

In certain embodiments, the subject has no detectable endogenous follicles. In certain embodiments, the subject has few detectable endogenous follicles. In certain embodiments, the subject is infertile.

In certain embodiments, the subject has a cancer. In certain embodiments, the subject is a human subject. In certain embodiments, the subject is receiving a cancer treatment. In certain embodiments, the subject has previously received a cancer treatment. In certain embodiments, the cancer is in remission. In certain embodiments, the subject is not receiving a cancer treatment due to the cancer remission. In certain embodiments, the cancer treatment is a chemotherapy or a radiation therapy. In certain embodiments, the cancer treatment has caused damage in ovarian tissue of the subject, and resulted in a dysfunction of the ovarian tissue.

In certain embodiments, the kits further include instructions for restoring reproductive health and/or fertility in a subject. In certain embodiments, the instructions include methods as described in Section 5.2 of the present disclosure.

The present disclosure also provides kits for treating an ovarian-associated disease, including a therapeutic component and an ovarian tissue derivative. Non-limiting exemplary ovarian-associated disease includes endometriosis, ovarian cysts, ovarian epithelial cancer, ovarian germ cell tumors, ovarian low malignant potential tumors, polycystic ovarian syndrome (PCOS), and premature ovarian failure (POF).

In certain embodiments, the ovarian tissue derivative includes a decellularized ovarian tissue. In certain embodiments, the decellularized ovarian tissue is derived from a mammal. In certain embodiments, the ovarian tissue derivative is selected from the ovarian tissue powder, ovarian tissue microparticles, ovarian tissue nanoparticles, ovarian tissue soluble fractions, ovarian tissue hydrogels, ovarian tissue matrix-bound nanovesicles, and combinations thereof.

Non-limiting exemplary therapeutic components that is selected from biomaterials, stem cells, therapeutic agents, and combinations thereof. Any suitable therapeutic components known in the art for treating or benefiting ovarian-associated diseases can be used with the presently disclosed kits.

Any suitable tools known in the art for obtaining follicles can be included in the kits disclosed herein. Non-limiting examples of tools for obtaining follicles include dissection tools (e.g., forceps, scissors), enzymatic reagents (e.g., collagenase, DNases), sterile filters, petri dishes, pipette, pipette tips, syringes, IVF dishes.

Any suitable tools known in the art for preparing and delivering the follicle-hydrogel mixture to the subject can be included in the kits disclosed herein. Non-limiting examples of tools for preparing and delivering the follicle-hydrogel mixture include tubes, syringes, needles (e.g., microinjection needles), and any tools that are required for minimally invasive procedures, such as transvaginal oocyte retrieval (TVOR) and amniocentesis.

In certain embodiments, the kits further include instructions for treating an ovarian-associate disease. In certain embodiments, the instructions include methods as described in Section 5.3 of the present disclosure.

### 8. EXAMPLE

The presently disclosed subject matter will be better understood by reference to the following Examples, which are provided as exemplary of the presently disclosed subject matter, and not by way of limitation.

### 8.1 Example 1: Bioengineering an In Situ Ovary (ISO) for Fertility Preservation

### 8.1.1 Introduction

Ovarian follicles are the major functional component of the ovary that produce hormones (e.g., estrogen) and mature eggs for ovulation. Chemotherapy or radiation treatments for cancer or other conditions can deplete the ovarian follicle pool, which can result in premature ovarian failure (POF), compromising ovarian hormone production and fecundity. Ovarian tissue cryopreservation is an option used to preserve the fertility of patients who cannot afford to delay gonadotoxic treatment. Upon remission, the ovarian cortex, which is rich in primordial follicles, can be transplanted back into patient survivors and ovulate eggs to establish natural pregnancies or be stimulated to produce eggs for in vitro fertilization (IVF). However, ovarian tissue transplantation remains an invasive surgical procedure and can be inappropriate in cases where there are concerns that ovarian tissues can harbor malignant cells (e.g., leukemia).

A major obstacle for restoring reproductive function and fertility can be the inability to accurately mimic the dynamic environment of the human ovary. Intraovarian transplantation can be used to generate live-births from infertile mice after the delivery of female germline stem cells. However, the existence and characterization of ovarian stem cells in the human adult ovary remains controversial. A previous attempt to restore fertility after chemotherapy using this approach resulted in follicle apoptosis.

The disclosed subject matter provides methods and devices to improve the effectiveness of intraovarian follicle transplantation, integration, and survival using a tissue-specific extracellular matrix (ECM) hydrogel. To obtain ECM hydrogels, tissues can be first be decellularized to remove immunogenic components. This process can be used to preserve an ovarian-specific acellular scaffold, which is composed of a unique profile of ECM proteins, proteoglycans, glycoproteins, and sequestered biomolecules (i.e., growth factors). However, currently available hydrogels fail to provide optimal environments for ovary. For example, although SDS is highly proficient at cellular ablation, acellular scaffolds prepared with SDS have led to adverse cytocompatibility, which has been directly linked to the disruption of matrix composition. The use of SDS to decellularize human ovarian medulla and cortical tissues, but observed low follicle recovery rates upon recellularization and xenotransplantation after three weeks.

The disclosed subject matter provides new decellularization methods using less abrasive detergents to obtain acellular scaffolds that can be processed into an ovarian-specific ECM (OECM) hydrogel to facilitate intraovarian follicle transplant. For example, the disclosed subject matter provides a method to enhance intraovarian microinjection of isolated immature follicles using an OECM hydrogel. Porcine ovaries were decellularized then characterized to evaluate the effects on ovarian tissue-specificity. Solubilized acellular ovaries were formed into OECM hydrogels to facilitate the delivery of primordial follicles and establish an in situ ovary (ISO). Chemotherapy-induced POF (ciPOF) mice were prepared using busulfan and cyclophosphamide to observe the impact on follicle populations. Intraovarian microinjection of immature follicles resuspended in OECM hydrogels was performed using ciPOF mice to examine the efficacy of this approach for donor follicle survival and reproductive outcomes. Transplanted ciPOF female mice were bred to fertile males and produced donor follicle (GFP+)-derived progeny. The disclosed subject matter offers a minimally-invasive method to support and enhance the transplantation of immature follicles to restore reproductive function in female cancer patients.

### 8.1.2 Materials and Methods

Ovarian tissue decellularization: Porcine ovaries from adolescent pigs (<1-year-old) were obtained from the local abattoir (Thoma Meat Market, Saxonburg, Pa.) and immediately stored on ice and frozen at −20° C. Ovaries were thawed, cleared of surrounding connective and adipose tissues, diced into cubes (˜0.125 cm³), and transferred to a flask containing cold Milli-Q water (MQ). The diced tissues were shaken manually with MQ until residual blood was visibly removed then replaced with fresh MQ and stored overnight at 4° C. The tissues were then rinsed in fresh MQ on an orbital shaker for 30 minutes at 300 rpm. The flask containing tissue was then replaced with a pre-warmed solution of 0.02% trypsin and 0.05% EDTA then agitated on a magnetic stir plate for one hour at 37° C. Ovaries were then rinsed three times with MQ for 15 minutes each at 300 rpm. A 3% solution of Triton X-100 was then added to the flask and shaken for one hour at 300 rpm. A subsequent wash cycle was implemented to remove any residual detergents from the tissues. Each wash cycle consisted of several distilled water rinses with manual shaking (until no bubbles were observed), then alternating washes of MQ and 1× PBS to neutralize and release detergent that was bound to the tissues. After the wash cycle was completed, the fluid was replaced with fresh MQ and stored overnight at 4° C. A 4% sodium deoxycholate solution was added to the flask and agitated for 1 hour at 300 rpm. A subsequent wash cycle was performed then the tissue was replaced with fresh MQ and stored overnight at 4° C. The ovarian tissues were then depyrogenated and disinfected with a 0.1% peracetic acid and 4% ethanol solution for two hours at 300 rpm. This step was followed by three rinses in MQ, 1× PBS, MQ for 15 minutes each at 300 rpm then stored in fresh MQ at 4° C. overnight. To ensure adequate removal of detergents and other chemical reagents, one final series of washes with MQ, 1× PBS, MQ were performed for 15 minutes each at 300 rpm. The decellularized tissues were then stored at −80° C. prior to lyophilization.

Characterization of decellularized tissues: Decellularized ovarian tissues were characterized using a number of qualitative and quantitative measures to verify the removal of genetic material and maintenance of ovarian specific proteins. Native and decellularized ovarian tissues were formalin-fixed, paraffin-embedded, sectioned, and stained using several histological procedures including DAPI (4′,6-diamidino-2-phenylindole), Hematoxylin and Eosin (H&E), and Periodic Acid-Schiff (PAS). Antibodies specific for ECM proteins Collagen I (Abcam, ab34710) and IV (Abcam, ab6586), Fibronectin (Abcam, ab23751), and Laminin (Abcam, ab11575) were evaluated using DAB (3,3′-Diaminobenzidine) immunohistochemistry (IHC) staining to show conservation after decellularization. IHC tissue sections were counterstained using Hematoxylin QS (Vector Labs, Cat No. H-3404) to show cell nuclei in contrast with resident ECM proteins. DNA removal was quantified using a PicoGreen dsDNA assay kit (Invitrogen, Cat No. P11496). A 2.5% agarose gel was used to detect DNA fragments at a resolution between 25 and 1000 bp. Hydroxyproline (HYP) and sulfated glycosaminoglycans (sGAG) assays were performed to detect collagen and sGAG content. Native and decellularized ovarian tissues were homogenized in a High Salt Buffer (pH 7.5, 50 mM Tris base, 150 mM NaCl, 5 M CaCl₂), 1% Triton-X-100, 1% Halt protease inhibitor cocktail, Pierce Biotechnology, Rockford, Ill.). Protein concentrations of the extracted tissues were determined using the BCA Protein Assay Kit (Pierce Biotechnology, Rockford, Ill.) and 50 ug total protein was used per sample for all assays. Ovarian specific growth factors including insulin growth factor (IGF-1) (R&D Systems, Minneapolis, Minn.), 17β-estradiol (R&D Systems, Minneapolis, Minn.), progesterone (Abcam, Cambridge, Mass.), anti-Müllerian hormone (AMH)(R&D Systems, Minneapolis, Minn.), and vascular endothelial growth factor (VEGF) (Abcam, Cambridge, Mass.), were quantified using enzyme-linked immunosorbent assays (ELISA).

Ovarian ECM digestion and hydrogel formation: Lyophilized ovarian ECM powder was solubilized via enzymatic digestion. A stock ECM digest concentration of 10 mg/mL was prepared by adding 200 mg of ECM powder to a 20 mL solution of pepsin (Sigma P7012) at a concentration of 1 mg/mL (≥2,500 units/mg) dissolved in 0.01N hydrochloric acids (HCl). Digestion was facilitated with an overhead mixer between 700-2000 RPM for less than 48 hours. Hydrogels were formed after neutralizing and buffering the solubilized ovarian ECM to physiological conditions. Two hydrogel concentrations (4 and 8 mg/mL) were prepared for testing and experimentation. A pre-gel solution was made on ice using the following components: (i) 10 mg/mL OECM digest stock (volume determined by desired final concentration) (ii) 0.1N NaOH ( 1/10th the volume of the digest), (iii) 10× PBS ( 1/9th the volume of the digest), and (iv) 1× PBS or L-15 medium (brought up to final volume). The solution was pulsed 3 times on a vortexer to mix then stored at 4° C. until further use.

Hydrogel characterization: Ovarian hydrogel ultrastructure was assessed using scanning electron microscopy (SEM). Hydrogels were fixed using 2.5% glutaraldehyde, washed with 1×PBS and post-fixed with osmium tetroxide (OsO4). Samples were washed again in 1×PBS to dilute the OsO4 then they were slowly exsiccated through a series of increasing ethanol concentrations. Complete dehydration was achieved using a critical point dryer. Dried hydrogels were sputter-coated with gold/palladium particles and imaged at 8,000× magnification. A proprietary Matlab code was used to analyze SEM images to determine various hydrogel fiber network characteristics. Ovarian hydrogel bulk viscoelastic properties were determined using a dynamic parallel plate (40 mm) rheometer (AR-2000 TA instruments). A time sweep (5% strain, 1 rad/s) was used to demonstrate the effect of ECM concentration on both the storage (G′) and loss (G″) modulus. Turbidimetric gelation kinetics were performed on the ovarian hydrogels.

Chemotherapy-induced POF model: Busulfan (Sigma) and cyclophosphamide (MP BioMedicals LLC) were combined to induce POF in 6-week old female mice (NCR nu/nu). Recipient mice were given a single intraperitoneal injection (IP) and allowed to recover up to 3 weeks prior to treatment. To initially identify the most appropriate chemotherapy regimen, four doses of busulfan/cyclophosphamide (mg/kg) were tested: (1) 12-100 (2) 12-200 (3) 24-100 (4) 24-200. Ovaries were excised, fixed in 4% paraformaldehyde (PFA), paraffin-embedded, and serial sectioned. Tissue sections were stained using Weigert's Hematoxylin Picric acid Methyl Blue then imaged under an upright bright field microscope. Every 10 sections were examined for total follicle number and classified by stage and quantified. The following criteria were used to count the follicles: (1) each follicle contains a visible oocyte (2) Primordial follicles have a single layer of squamous granulosa cells (3) Primary follicles have a single layer of cuboidal granulosa cells (4) Secondary follicles contain two to four layers of granulosa cells without the development of an antrum (5) Antral follicles have greater than four layers of granulosa cells as well as definitive antrum. The total number of follicles were quantified then the sum was multiplied by 10 to provide an estimate of the entire follicle population of each ovary.

Enzymatic follicle isolation: Ovarian donor follicles were prepared using a physical and enzymatic isolation procedure. First, ovaries were excised from 6-14 day old female (DBA GFP/nu) mice and placed into pre-warmed L-15 (Leibovitz's) medium. The ovaries were freed from the bursa using a pair of forceps and an insulin needle. Ovaries were then minced using insulin needles into small fragments to aid in digestion. The ovarian fragments were then added to 1.5 mL microcentrifuge tube containing 500 μL of pre-warmed L-15 medium and 50 μL of Liberase™ (13 Winsch units/mL). The tubes were then placed on an orbital shaker and agitated at 200 rpm for 5 minutes at 37° C. After incubation, the mixture was then pipetted gently for one minute to help free the ovarian follicles from connective tissue. This process was repeated once more until the ovaries had been completely dissociated. After digestion, 10% fetal bovine serum was added to the mixture to halt enzyme activity. The tubes were placed in an upright position for 15 minutes at 37° C. to allow follicles to sediment. After 15 minutes, 200 μL of the mixture was carefully pipetted off the top to remove singular ovarian cells. The samples were centrifuged at 100 g for 5 minutes to loosely concentrate the follicles. A syringe needle was used to gently remove the medium from the tube without disturbing the follicle pellet. Finally, the pellet was resuspended in a chilled 4 mg/mL OECM pre-gel and kept on ice in preparation for follicle microinjection.

In vivo follicle microinjection: Eight-week-old ciPOF female mice (NCR nu/nu) mice were anesthetized and placed on the operating table with their back exposed. A single midline dorsal incision (0.5 cm) was made using small scissors. Subcutaneous connective tissue was then freed from the underlying muscle on each side using blunt forceps. Once the ovary was located under the thin muscle layer, a small incision (<1 cm) was made to gain entry to the peritoneal cavity. The edge of the incision was held with tooth forceps, while the ovarian fat pad was removed to expose the ovary and surrounding bursa. A small volume (<10 μL) of chilled follicle-OECM pre-gel was transferred to a glass needle (filament Cat #: FB245B and borosilicate glass micropipette Cat #: B100-75-10) then secured to a pressurized microinjection system. Eppendorf microinjection system (TransferMan NK2 and FemtoJet) was used for follicle delivery and surgical manipulation observed under a Nikon SMZ stereomicroscope. The loaded needle was positioned perpendicular to the ovary and guided into the ovarian cortex, where the follicle-OECM mixture was slowly injected at a constant pressure ranging from 50-250 hPa. For each injection, approximately 1.0×10³ follicles were transplanted. The same surgical and injection procedures were performed contralaterally. The follicle-injected ovaries were placed back into the abdominal cavity, the muscle layers were sutured, and the skin incision was stapled.

Mating: Two weeks after injection both the follicle recipient and non-injected control ciPOF nude female mice were bred to male nude mice (NCR nu/nu, Taconic). The breeding was conducted for three cycles, which concluded at 106 days on average. Pups born were fostered within 1 day with NCR nu/+(Taconic) females due to the lack of developed mammary glands in the nude mouse strain used for recipients, then they were weaned at 3-4 weeks old. Pups (DBA-GFP/nu−, Orwig Lab) inherited from the follicle injected recipients were selected based on physical traits consisting of fur, dark eyes, or GFP expression. Genotyping was performed using mouse tail DNA and standard PCR with the following primers: GFP forward primer sequence: GAA CGG CAT CAA GGT GAA CT (SEQ ID NO: 1); GFP reverse primer sequence: TGC TCA GGT AGT GGT TGT CG (SEQ ID NO: 2); β-actin forward primer sequence: CGG TTC CGA TGC CCT GAG GCT CTT (SEQ ID NO: 3); β-actin reverse primer sequence: CGT CAC ACT TCA TGA TGG AAT TGA (SEQ ID NO: 4); primers prepared by Integrated DNA Technologies, Inc.). PCR products were run on a 2.5% agarose gel and imaged under UV light. The resulting pups were grown to 8 weeks and bred for fertility status. Second generation breeding pairs consisted of a GFP/nu-experimental female and DBA/2 control male (Jackson), a GFP/nu− experimental female and GFP/nu− experimental male, and a DBA/2 control female and GFP/nu-experimental male. Breeding pairs were separated after two weeks.

Immunofluorescence staining and imaging of microinjected ovaries: Ovaries were excised at the conclusion of the third breeding cycle and fixed in 4% PFA overnight then embedded in paraffin. Tissues were serial sectioned and stained with DAPI to evaluate the presence of transplanted GFP+ follicles. Four ovaries were evaluated per treatment group. Nikon Eclipse Ti inverted microscope and NIS Elements software was used to capture representative images of GFP positive structures within each tissue section. DAPI and FITC channels were taken separately and merged to demonstrate the population of cells expressing GFP.

Statistical analysis: All data were expressed as mean±s.e.m and plotted using GraphPad Prism 7.02. For the analysis of normally (parametrically) distributed data, the individual means were compared using an unpaired, two-tailed, t-test. For the analysis of non-parametrically distributed data, the mean ranks were compared using an unpaired, one-way ANOVA (Kruskal-Wallis) with adjusted P-values calculated based upon Dunn's multiple comparisons test. Exact P-values resulting from the statistical analysis are presented within each figure.

### 8.1.3 Results

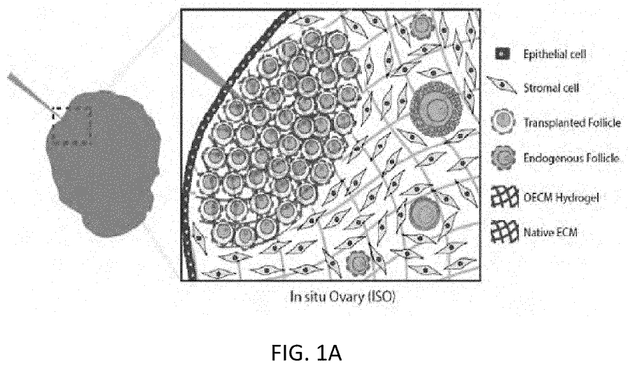

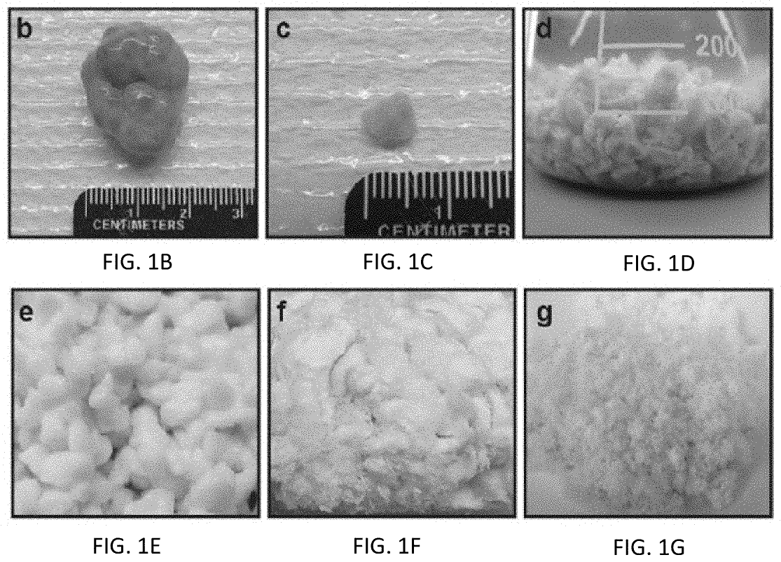

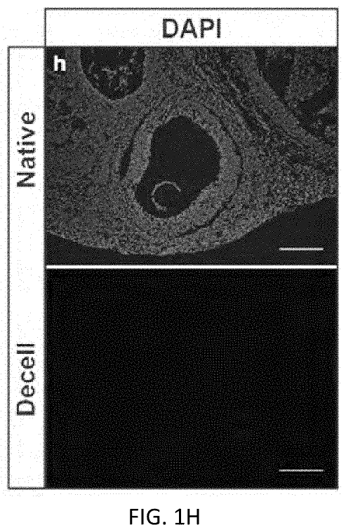

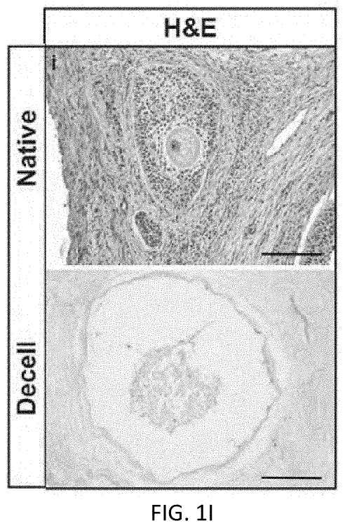

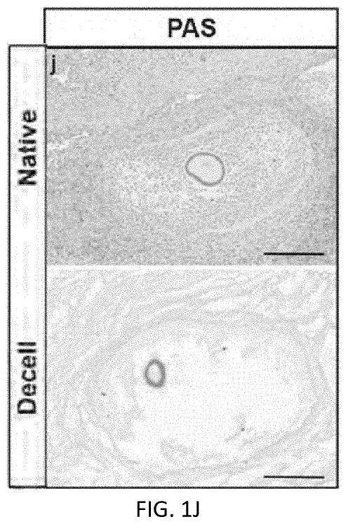

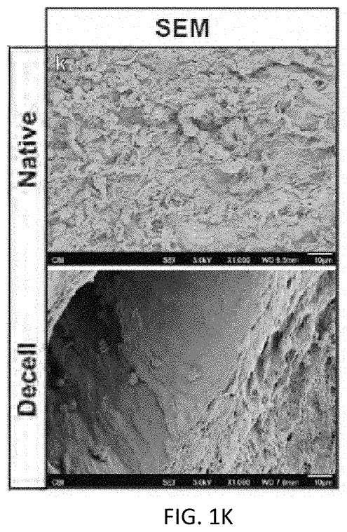

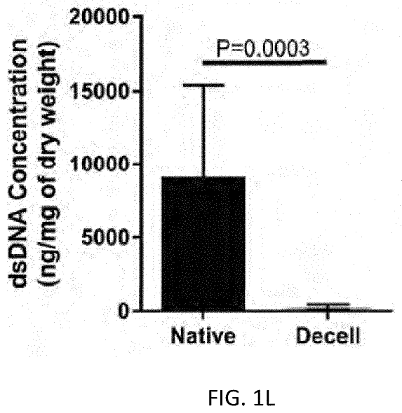

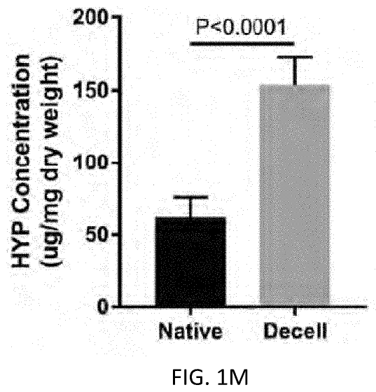

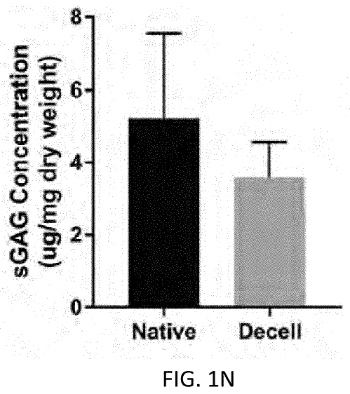

Biomaterial selection and tissue processing: The damaging effects of chemotherapy on ovarian tissues significantly reduces a patient's follicle population, which has a direct impact on fertility and endocrine function. In addition, chemical treatments have been implicated in the microvasculature and stromal cell irregularities culminating in a compromised environment for cell survival. These unfavorable conditions cause a depletion of ovarian follicles and may reduce follicle viability post-transplantation. Therefore, to re-establish the ovarian tissue microenvironment and repopulate the depleted endogenous follicle pool, an ISO was bioengineered using an OECM hydrogel to facilitate intraovarian follicle transplant and provide a temporary niche to aid follicle engrafiment and survival (FIG. 1A). FIG. 1A is a graphical representation that shows methods for restoring fertility in patients with chemotherapy-induced premature ovarian failure (ciPOF). Intraovarian microinjection of an ovarian-specific ECM (OECM)hydrogel can support the delivery and long-term survival of exogenous primordial follicles within an in situ ovary (ISO). The damaged ovarian tissue primarily includes stromal cells and a depleted population of endogenous follicles.

To prepare acellular ovarian scaffolds, porcine ovaries (FIG. 1B) were diced (FIG. 1C) then processed using a series of enzymatic and detergent-based washes to remove immunogenic material (FIG. 1D). Young (<1-year-old) porcine ovaries were sourced for decellularization. Ovaries were diced into small cubes (˜0.125 cm3). Cubed ovaries were added to a flask and decellularized using enzymes and detergents. Trypsin and EDTA were used in tandem to disrupt cell adhesions to the ECM prior to treatments with Triton X-100 and sodium deoxycholate, which act to permeabilize cell membranes. Tissues transitioned from an initial opaque appearance to translucent at the conclusion of the decellularization steps (FIG. 1E). Decellularized ovarian tissues appeared white. Intermediate water washing steps proved to be critical for the complete removal of residual cells and detergent from the tissues. Once the tissues were completely decellularized, they were frozen, lyophilized (FIG. 1F), and milled into a powder (FIG. 1G) prior to biochemical testing and downstream processing. Ovaries were frozen, then lyophilized to remove their water content, and powdered OECM was prepared using a mill. To demonstrate the effective removal of immunogenic components and preservation of extracellular matrix (ECM) components, a set of histological stains and biochemical assays were performed. Fluorescence staining with 4′,6-diamidino-2-phenylindole (DAPI) showed few if any nuclei present within the decellularized tissues in comparison to native ovarian tissue controls (FIG. 1H). H&E (FIG. 1I) and PAS (FIG. 1J) staining showed clear retention of ovarian microarchitectures, such as structural aspects of follicles, zona pellucida, and corpora lutea, while sparse cellular content was visible. Native (top row) and decellularized (bottom row) images of DAPI (200 μm scale), H&E (100 μm scale), and periodic acid-Schiff (PAS) (100 μm scale) staining determined that decellularized tissues removed cellular content while preserving ovarian tissue morphology.

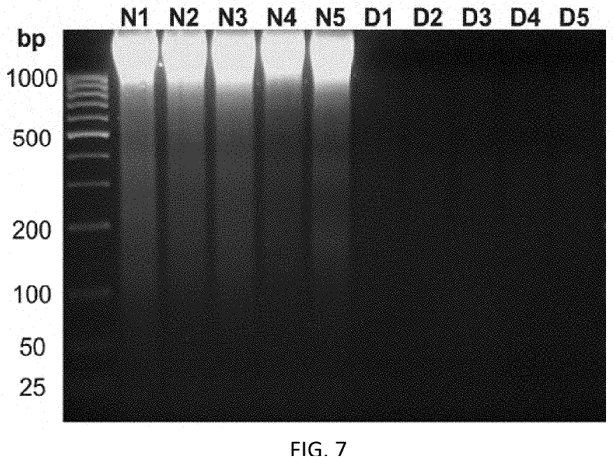

This highlights the effectiveness of the disclosed method to successfully remove cellular content while causing limited disruption of tissue-specific morphology. Scanning electron micrographs (SEM) further detailed the dense cellular content within native ovarian tissues, whereas decellularized tissues appeared to show vacated follicular compartments surrounded by a porous scaffold (FIG. 1K). Scanning electron micrographs (SEM) show a dense cellular ultrastructure in native ovaries in comparison to a porous decellularized scaffold (10 μm scale). PicoGreen assay indicated that decellularized ovarian tissues significantly reduced the dsDNA concentration. Data represent mean±s.e.m. of ng/mg dry weight. A PicoGreen assay demonstrated greater than 98% reduction of dsDNA between native (9126±1988 ng/mg) and decellularized (262.4±59.96 ng/mg) samples (FIG. 1L). Gel electrophoresis further showed a lack of DNA (FIG. 7) within the decellularized tissues in comparison to native controls, suggesting a reduced potential for disease transmission and adverse immune reaction to cellular contents. Collagen and sulfated glycosaminoglycans (sGAG) were also examined to determine their retention post-decellularization. A hydroxyproline (HYP) assay was used to estimate the total collagen content within the scaffold. Native tissues (61.95±6.064 ug/mg) contained significantly less HYP as a percentage of dry weight than decellularized tissues (153.3±8.564 ug/mg) due to the loss of cellular mass; however, under this assumption, the total collagen content within the decellularized scaffold as a fraction of the dry weight of all components was enriched after decellularization (FIG. 1M). Hydroxyproline (HYP) concentration was significantly enriched in decellularized tissues. sGAG content was also preserved with no significant difference observed between native (5.24±1.03 ug/mg) and decellularized (3.59±0.436 ug/mg) samples (FIG. 1N). Sulfated-Glycosaminoglycans (sGAG) levels did not differ significantly between native and decellularized samples.

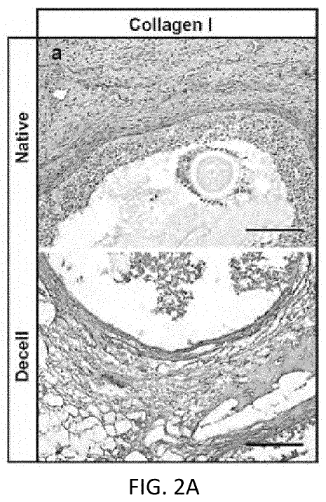

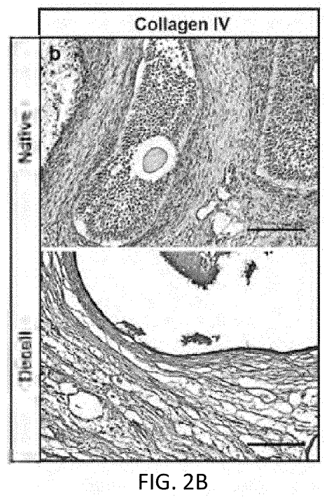

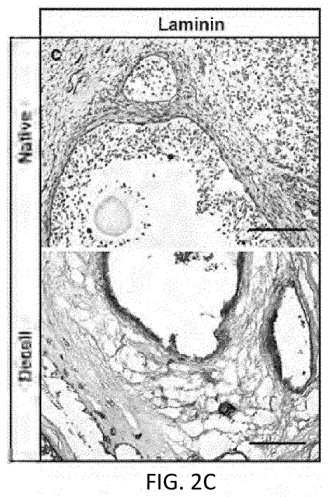

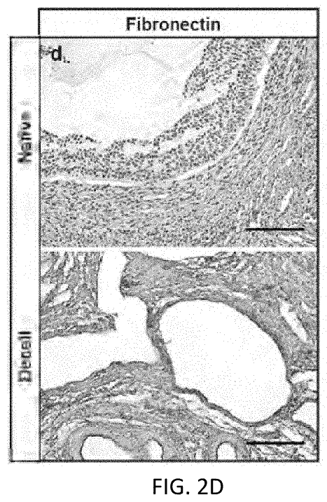

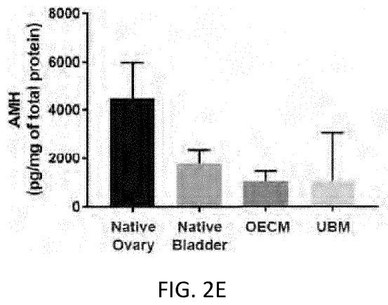

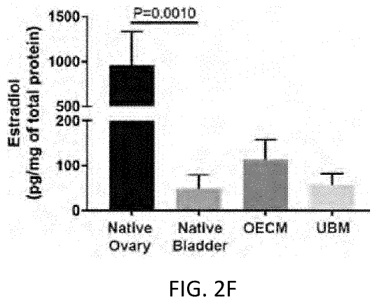

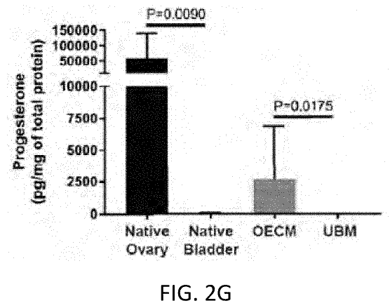

Ovarian tissue specificity present post-decellularization: The ECM is composed of a tissue-specific milieu of secreted proteins and proteoglycans that support the desired functions of a tissue. In the ovary, the ECM undergoes dynamic remodeling throughout the reproductive life span and is essential for regulating folliculogenesis and ovulation. OECM provides mechanical support, maintains normal cell morphology, promotes cell proliferation, and steroidogenesis. Additionally, the OECM can sequester hormones and growth factors within the follicle niche to facilitate paracrine and endocrine signaling. Therefore, the retention of OECM proteins can be ideal for supporting follicles within the ISO. To determine the effects of decellularization on ECM retention, a subset of the most highly expressed OECM proteins is characterized: Collagen I, Collagen IV, laminin, and fibronectin. Immunohistochemistry revealed that collagen I was distributed uniformly in the native samples, with a slight enrichment surrounding the thecal compartments of the follicles in decellularized samples (FIG. 2A). Collagen I was uniformly expressed throughout each of the tissues. Collagen IV was also labeled, showing definitive staining within the basement membrane of the epithelial layer and the basal lamina of individual follicles in both the native and decellularized groups (FIG. 2B). Collagen IV appeared to concentrate in the basal lamina of the follicles, and the basement membrane surrounds the epithelial layer of the ovary. Similar to Collagen IV, laminin was predominantly found within the basal lamina adjacent to the theca interna surrounding follicles (FIG. 2C). Laminin was present within the thecal compartment. Finally, fibronectin appeared to be conserved throughout the ovarian tissues with little to no differences in distribution noted between the native and decellularized groups (FIG. 2D). Fibronectin was evenly expressed in lower concentrations.

Ovarian hormones and growth factors sequestered in the OECM orchestrate both local and systemic endocrine function. The hypothalamic-pituitary-gonadal (HPG) axis stimulates the production of ovarian hormones, which act to modulate hormone production in a cyclic manner. The hypothalamus produces gonadotropin-releasing hormone (GnRH), which stimulates the secretion of follicle-stimulating hormone (FSH) and luteinizing hormone (LH). FSH and LH trigger the production of estradiol, follicle development, and ovulation. Estradiol from the ovulatory follicle and progesterone from the resulting corpus luteum provide feedback to either inhibit or stimulate hormone secretion from the hypothalamus and pituitary. Spatiotemporal production of these reproductive hormones primarily facilitates follicle development, ovulation, and pregnancy.

Certain hormones produced by the ovary were evaluated due to their roles in follicle selection. For example, anti-Müllerian hormone (AMH), estradiol, and progesterone were evaluated. AMH is produced by granulosa cells of pre-antral and antral follicles. As AMH levels increase, it can inhibit the recruitment of primordial follicles and decrease the responsiveness of large pre-antral/antral follicles to FSH. AMH is one of the few hormones that are produced during the early stages of folliculogenesis, which are recognized as gonadotropin-independent. Estradiol is also produced by follicular cells and is most commonly known for its role in the LH surge, which triggers ovulation; however, at low concentrations, estradiol can function as a negative regulator of FSH, which inhibits follicle growth. The corpus luteum, which arises from the cells of ovulatory follicles and is present during the late luteal phase, produces high levels of progesterone, which is necessary for maintaining pregnancy. Like estradiol, progesterone can also inhibit FSH production further delaying follicle growth.

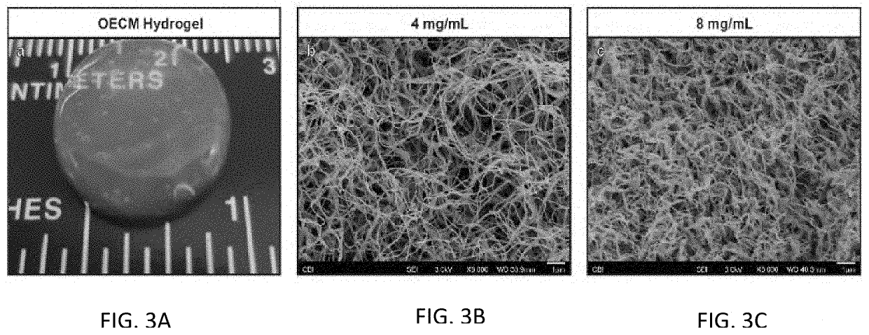

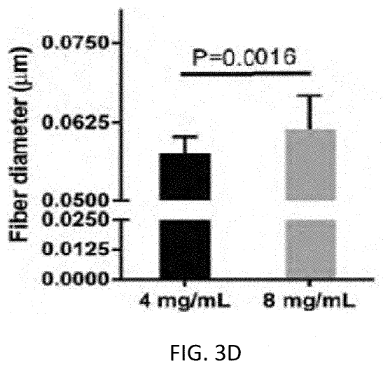

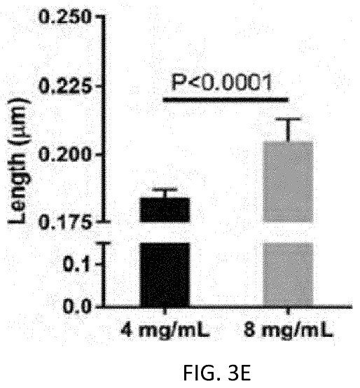

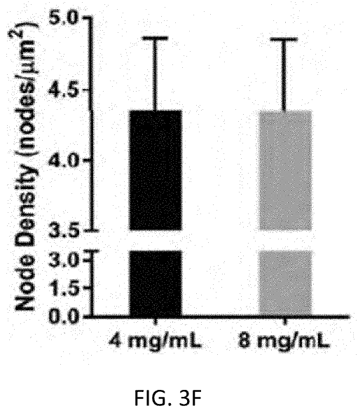

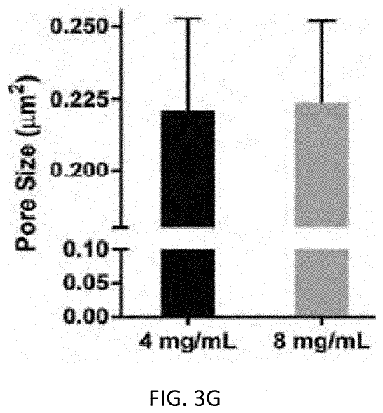

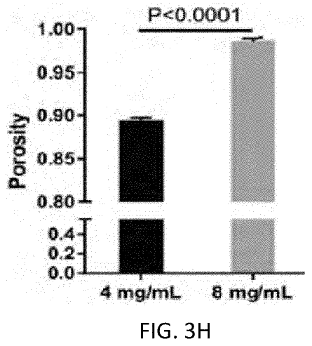

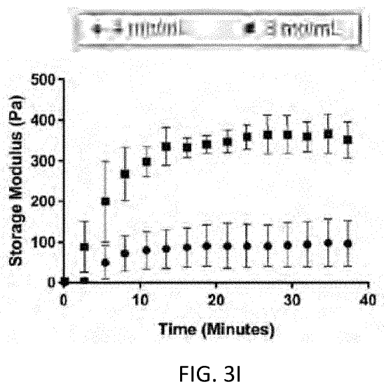

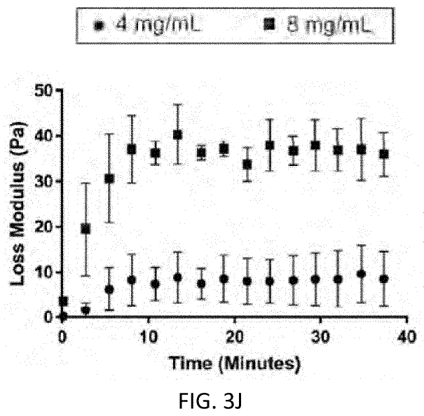

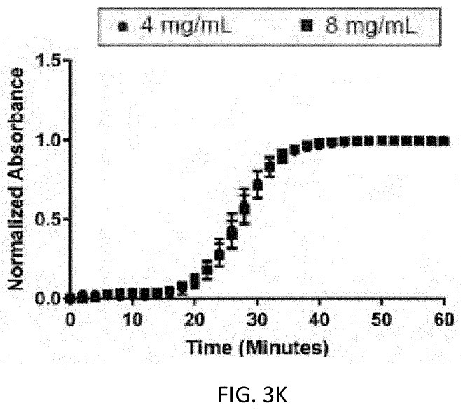

The identified ovarian hormones were involved in the survival of transplanted immature follicles within the disclosed ISO. To elucidate the effects of decellularization on the disruption of these components, a large batch of 100 ovaries were separated into five groups (n=20 per group) and analyzed as independent samples. As controls, both native and decellularized urinary bladder matrix (UBM), collected from female pigs and prepared as previously described, were used to determine if there were significant differences in the hormone concentrations based upon tissue source. After tissue homogenization, the protein was extracted from each group and tested using biochemical assays for AMH, estradiol, and progesterone. ELISA quantification determined that decellularized OECM samples contained low concentrations of each of the ovarian-specific analytes: AMH (1031±192.9 pg/mg), estradiol (113.8±19.63 pg/mg) and progesterone (2697±1890 pg/mg) (FIG. 2E-G). Anti-Müllerian hormone (AMH) was measured at high concentrations within native ovarian tissues but was reduced by >50% within decellularized OECM. Estradiol concentrations were significantly higher in the native ovary in comparison to the native bladder, and OECM was two-fold greater than UBM. Progesterone levels were significantly greater in both native ovary and OECM in comparison to native bladder and UBM, respectively. ELISAs were conducted using 100 ovaries batched into 5 independent samples (n=20 ovaries). Native ovaries contained significantly higher levels of estradiol and progesterone when compared to the native bladder. Furthermore, decellularized OECM had significantly higher progesterone values than UBM. Additional analytes associated with follicle development, insulin-growth factor (IGF-1), and vascular endothelial growth factor (VEGF), were also tested but were undetectable within the decellularized samples. Ovarian hydrogel properties modified with changes in ECM concentration: Once the decellularized tissues were processed and characterized, the OECM was solubilized. Then the digested material was neutralized to prepare the hydrogels. Visibly transparent hydrogels were formed after exposure to physiological conditions for approximately 20 minutes (FIG. 3A). Solubilized ovarian ECM (OECM) formed transparent hydrogels upon neutralization and exposure to physiologic conditions. SEM was used to evaluate the hydrogel ultrastructural properties at 4 and 8 mg/mL ECM concentrations (FIGS. 3B and 3C). Fiber network characteristics were quantified using SEM imaging and digital image analysis with concentration-dependent effects observed with a significant increase in fiber diameter, fiber length, and bulk porosity in the 8 mg/mL OECM hydrogel group (FIG. 3D-H). These results indicate that individual fiber and large-scale network properties such as the bulk porosity are dependent on ECM concentration.

To determine the viscoelastic properties of the OECM hydrogel, a rheological time sweep was performed on varying ECM concentrations. An increase in ECM concentration from 4 to 8 mg/mL appeared to correlate with elevated storage (G′) and loss (G″) moduli (FIGS. 3I and 3J). However, there were no observable differences in turbidimetric gelation kinetics with a change in ECM concentration (FIG. 3K). Gelation time varied based upon the test conditions. Specifically, direct conduction with the Peltier plate achieved complete gelation approximately 15 minutes prior to the samples heated via convection during gelation kinetics testing. However, once gelation initiated, hydrogels from both test formats consistently solidified within 20 minutes.

Alkylating agents significantly reduce endogenous follicle population: After developing and characterizing the OECM hydrogel as a carrier for follicle injection, a clinically relevant ciPOF mouse model was created. POF was induced using alkylating agents, busulfan, and cyclophosphamide. Briefly, a single intraperitoneal (IP) injection was given to 6-week old nude female mice. Dosing was titrated to determine an appropriate treatment that can significantly reduce the endogenous follicle pool to lower or eliminate the chances of fertility. The following doses were tested, abbreviated as busulfan-cyclophosphamide (mg/kg): (1) 12-100 (2) 12-200 (3) 24-100 (4) 24-200.

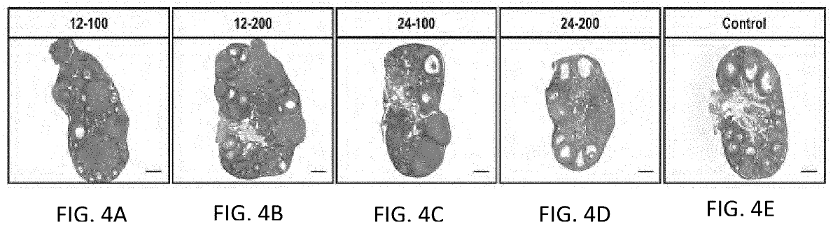

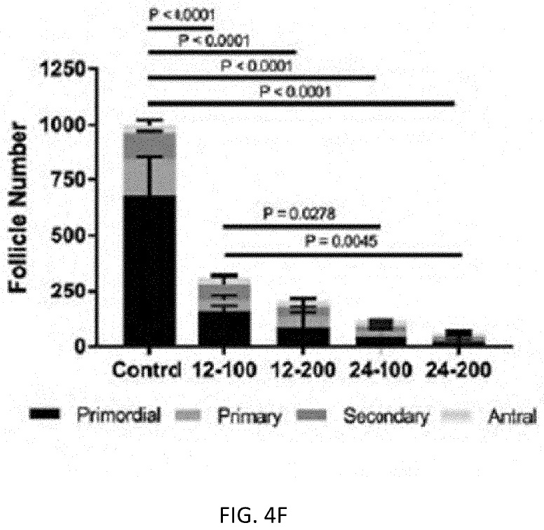

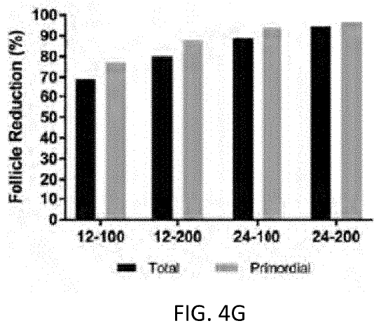

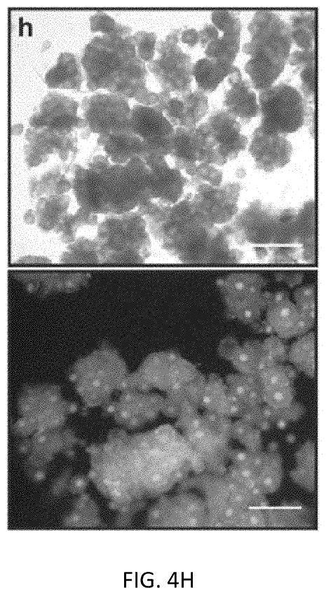

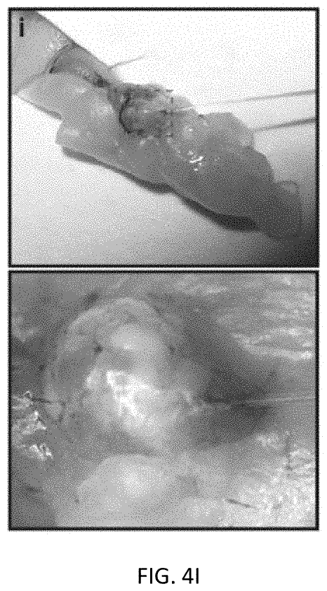

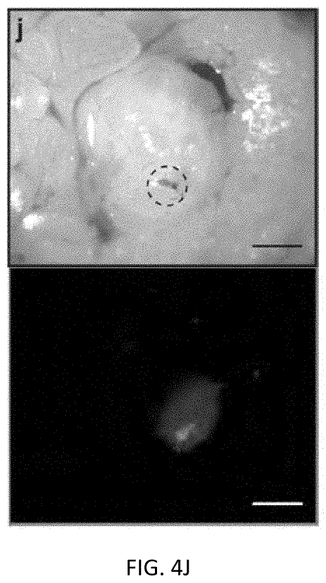

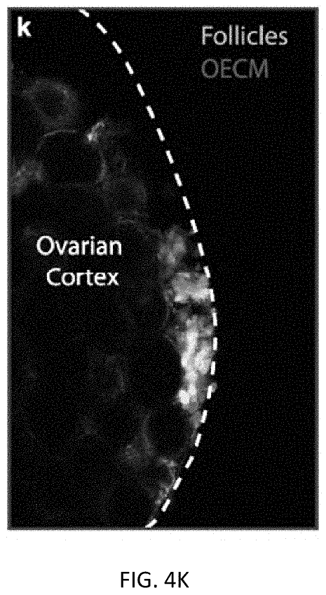

At three weeks of post-IP injection, histological staining with Weigert's Hematoxylin-Picric Methyl Blue clearly illustrated the damaging effects of each chemotherapy regimen on the follicle population within the ovaries. Dose-dependent effects were observed with elevated levels of busulfan and cyclophosphamide reducing the follicle numbers (FIGS. 4A-4F). 12-100 mg/kg, 12-200 mg/kg, 24-100 mg/kg, 24-200 mg/kg, and non-injected (control) were administered via single IP injection. Ovaries were excised at 3 weeks post-treatment and stained using Weigert's Hematoxylin Picric Acid Methyl Blue. Scale, 250 μm. Follicles were manually counted, quantified, and classified by the developmental stage showing a steady decline of the total follicle number with increasing dose. Additionally, busulfan appeared to have an enhanced effect on follicle depletion in comparison to cyclophosphamide.

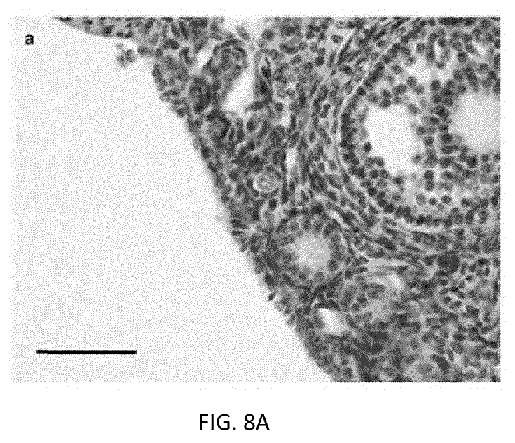

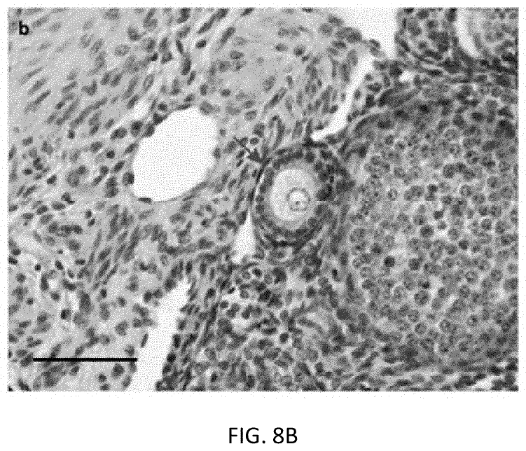

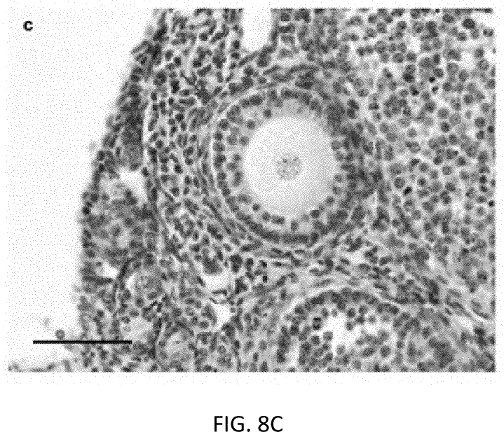

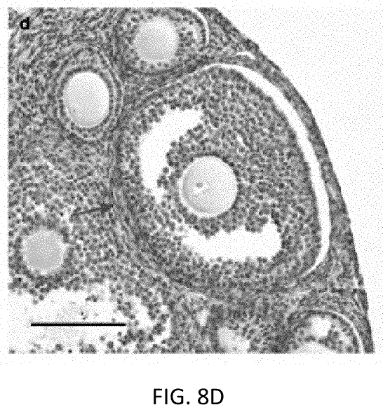

Follicles were counted based upon the developmental stage (FIGS. 8A-8D and Table I). Primordial follicles were recognized by a central oocyte surrounded by a single layer of squamous granulosa cells. Primary follicles were counted if they contained a single oocyte with a layer of cuboidal granulosa cells. Secondary follicles contained an oocyte with 2-4 layers of cuboidal granulosa cells. Antral follicles were distinguished by an oocyte with several layers of cuboidal granulosa cells containing pockets of antral fluid. Arrows indicate counted follicles.

TABLE I
Follicle Counts Post-Chemotherapy

| | Control | 12-100 | 12-200 | 24-100 | 24-200 |
|---|---|---|---|---|---|
| Follicle Stage | (n = 2) | (n = 5) | (n = 5) | (n = 4) | (n = 4) |
| Primordial | 680.0 | 156.0 | 84.0 | 42.5 | 20.0 |
| Primordial SD | 169.7 | 25.1 | 67.3 | 44.3 | 27.1 |
| Primary | 160.0 | 50.0 | 48.0 | 22.5 | 5.0 |
| Primary SD | 14.1 | 22.4 | 44.4 | 9.6 | 5.8 |
| Secondary | 125.0 | 74.0 | 44.0 | 25.0 | 17.5 |
| Secondary SD | 7.1 | 35.8 | 39.1 | 12.9 | 15.0 |
| Antral | 35.0 | 30.0 | 24.0 | 22.5 | 15.0 |
| Antral SD | 21.2 | 15.8 | 11.4 | 5.0 | 12.9 |
| Total | 1000.0 | 310.0 | 200.0 | 112.5 | 57.5 |
| Total SD | 155.6 | 53.4 | 121.0 | 61.3 | 48.6 |
| % Reduction (Total) | | 69.0% | 80.0% | 88.8% | 94.3% |
| % Reduction | | 77.1% | 87.6% | 93.8% | 97.1% |
| (Primordial Follicles) | | | | | |

Table I shows follicle count post-chemotherapy. The table lists the means and standard deviations (SD) of quantified follicles at each of the tested dosages (busulfan-cyclophosphanmide mg/kg). Total and primordial follicle reductions were calculated as a percentage of the follicles present within healthy control mice.

Primordial and primary follicles were significantly reduced after exposure to all treatments in comparison to non-treated control mice (FIG. 4G and Table II). Primordial follicles reduction in both the 24-100 and 24-200 is indicative of severe depletion of the ovarian reserve, significantly lowering potential fertility and could represent premature ovarian failure (POF).

TABLE II
Dose-dependent Statistical Differences in Follicle Counts
After Chemotherapy.

| Follicle | | | | |
|---|---|---|---|---|
| Stage | Group Comparison | Significant? | Summary | P-Value |
| Primordial | Control vs. 12-100 | Yes | **** | <0.0001 |
| | Control vs. 24-100 | Yes | **** | <0.0001 |
| | Control vs. 12-200 | Yes | **** | <0.0001 |
| | Control vs. 24-200 | Yes | **** | <0.0001 |
| | 12-100 vs. 24-100 | No | ns | 0.0955 |
| | 12-100 vs. 12-200 | No | ns | 0.3891 |
| | 12-100 vs. 24-200 | Yes | * | 0.0352 |
| | 24-100 vs. 12-200 | No | ns | 0.8517 |
| | 24-100 vs. 24-200 | No | ns | 0.9846 |
| | 12-200 vs. 24-200 | No | ns | 0.5538 |
| Primary | Control vs. 12-100 | Yes | ** | 0.0013 |
| | Control vs. 24-100 | Yes | *** | 0.0002 |
| | Control vs. 12-200 | Yes | ** | 0.0011 |
| | Control vs. 24-200 | Yes | **** | <0.0001 |
| | 12-100 vs. 24-100 | No | ns | 0.5468 |
| | 12-100 vs. 12-200 | No | ns | >0.9999 |
| | 12-100 vs. 24-200 | No | ns | 0.1333 |
| | 24-100 vs. 12-200 | No | ns | 0.6133 |
| | 24-100 vs. 24-200 | No | ns | 0.8779 |
| | 12-200 vs. 24-200 | No | ns | 0.1611 |
| Secondary | Control vs. 12-100 | No | ns | 0.2642 |
| | Control vs. 24-100 | Yes | ** | 0.0086 |
| | Control vs. 12-200 | Yes | * | 0.03 |
| | Control vs. 24-200 | Yes | ** | 0.0048 |
| | 12-100 vs. 24-100 | No | ns | 0.1349 |
| | 12-100 vs. 12-200 | No | ns | 0.4933 |
| | 12-100 vs. 24-200 | No | ns | 0.0679 |
| | 24-100 vs. 12-200 | No | ns | 0.859 |
| | 24-100 vs. 24-200 | No | ns | 0.9957 |
| | 12-200 vs. 24-200 | No | ns | 0.6543 |
| Antral | Control vs. 12-100 | No | ns | 0.9899 |
| | Control vs. 24-100 | No | ns | 0.7998 |
| | Control vs. 12-200 | No | ns | 0.8471 |
| | Control vs. 24-200 | No | ns | 0.4231 |
| | 12-100 vs. 24-100 | No | ns | 0.9076 |
| | 12-100 vs. 12-200 | No | ns | 0.9465 |
| | 12-100 vs. 24-200 | No | ns | 0.4535 |
| | 24-100 vs. 12-200 | No | ns | 0.9998 |
| | 24-100 vs. 24-200 | No | ns | 0.9222 |
| | 12-200 vs. 24-200 | No | ns | 0.8377 |
| Total | Control vs. 12-100 | Yes | **** | <0.0001 |
| | Control vs. 24-100 | Yes | **** | <0.0001 |
| | Control vs. 12-200 | Yes | **** | <0.0001 |
| | Control vs. 24-200 | Yes | **** | <0.0001 |
| | 12-100 vs. 24-100 | Yes | * | 0.0278 |
| | 12-100 vs. 12-200 | No | ns | 0.3087 |
| | 12-100 vs. 24-200 | Yes | ** | 0.0045 |
| | 24-100 vs. 12-200 | No | ns | 0.5743 |
| | 24-100 vs. 24-200 | No | ns | 0.8932 |
| | 12-200 vs. 24-200 | No | ns | 0.1547 |

Table II displays statistical differences between follicle counts based upon chemotherapy treatment. One-way ANOVA with Tukey's multiple comparisons showed significant differences (highlighted in green) within each follicle stage and total follicles.

The outcomes of chemotherapy titration suggested that a range of doses can impair fertility outcomes or lead to ovarian insufficiency. The efficacy of hydrogel-assisted follicle microinjection was evaluated using female mice treated across each dosage of chemotherapy. However, the 24-100 dosage was selected as the primary treatment to prepare ciPOF mouse model, which represents a likely candidate to develop POF due to the significant loss of follicles.

Enzymatic follicle isolation and microinjection provide an efficient transplant procedure: Follicle incubation time from isolation to transplant is a concern for cell therapy applications as it can directly impact viability. Therefore, an enzymatic isolation protocol was used to reduce the time needed to obtain a large pool of immature follicles for transplant. To enable the identification of transplanted versus endogenous follicles, follicles were isolated from transgenic mice exhibiting ubiquitous GFP under the chicken β-actin (CAG) promoter. Enzyme concentrations of 10% and 20% were evaluated for both Liberase™ and DH (13 Wünsch units/mL) to determine their effects on follicle disaggregation and quality. Briefly, Liberase™™ or DH was added to the minced ovaries, then two five-minute cycles of physical agitation at 37° C. were performed with a minute of pipetting after each cycle. After assessing each sample, the formulation with the 10% Liberase™ showed improved results, which released a large population of morphologically normal GFP follicles during the 12-minute isolation procedure (FIG. 4H). In order to estimate the number of follicles isolated with this procedure, follicles were manually counted using a hemocytometer. There were approximately 1.5×10³ total follicles isolated per ovary with 74.4% of this population identified at the primordial stage.

The efficacy of using microinjection to transplant follicles into the ovarian cortex of ciPOF mice was evaluated. Immature follicles naturally reside within the ovarian cortex, as this region of the ovary has mechanically distinct properties that support the early stages of folliculogenesis. Therefore, precisely dispense of follicles into or near the cortex was performed. Intragonadal cell delivery was used to facilitate intraovarian follicle transplants to establish an ISO. First, the delivery of the OECM hydrogel alone via microinjection was performed. A small volume of hydrogel was injected into the ovarian cortex, and the recipient animal was sacrificed to visualize the injection site (FIG. 4I). Brightfield images show the gross morphology of the ovary during microinjection (top) and magnified to show the injection site for follicle transplant (bottom) (FIG. 4I). Bright-field images clearly illustrated Trypan blue dye at the site of injection at the tissue surface (FIG. 4J). Pressurized microinjection was tested as a potential technique to deliver the OECM hydrogel (TRITC-labeled) and visualized under bright field (top) and fluorescence (bottom). Furthermore, the use of a TRITC-labeled OECM hydrogel allowed to identify the hydrogel at the ovarian surface post-injection (FIG. 4J). These results confirmed this method as a suitable delivery mechanism for the viscous OECM within a specific anatomical location of the ovary. The same evaluation with the addition of isolated GFP follicles was performed to determine if the gel and follicles could be delivered simultaneously, resulting in the formation of an ISO. Ovaries excised from the TRITC-OECM hydrogel and GFP follicle microinjection clearly indicated a co-localization of the OECM and follicles within the ovarian cortex (FIG. 4K). Multiphoton images confirmed that intraovarian follicle microinjection of the isolated GFP follicles and OECM co-localized within the ovarian cortex to form an in situ ovary. The dotted line (white) indicates the outer surface of the ovarian epithelium. Microinjected follicles give rise to multiple generations of GFP pups: the disclosed ciPOF mouse model (24-100 mg/kg) was used to investigate the therapeutic potential of an ISO to support follicle survival after intraovarian microinjection. Approximately 1.0×10³ GFP+ follicles resuspended in 8 μL of OECM hydrogel were microinjected into the ovarian cortex of ciPOF nude female mice (n=2). Non-injected ciPOF nude female mice were used as controls (n=2). To reduce tissue damage due to needle puncture, only a single follicle injection was performed on each ovary. Freshly isolated GFP follicles from 6-14 day female (DBA-GFP/nu−) mice were used to ensure a predominantly immature follicle population at the time of injection.

To determine the effects of follicle microinjection on fertility, follicle recipient, and non-injected control ciPOF nude female mice were mated with nude male mice for three breeding cycles (˜100 days). The breeding strategy was designed to definitively distinguish between pups derived from transplanted or endogenous follicles (Table II).

TABLE III
Breeding Strategy

| | | Female | | | |
|---|---|---|---|---|---|
| | | Endogenous Nude Mouse Follicles | | Transplanted GFP Mouse Follicles | |
| | | Nu− | Nu− | DBA GFP+ | DBA GFP− |
| Male | Nu− | NCR nu−/nu−: | NCR nu−/nu−: | GFP+/nu−: | GFP−/nu−: furry, |
| | | nude mice | nude mice | furry, dark | dark eyes, does |
| | | with red eyes | with red eyes | eyes, glows | not glow green |
| | | | | green under UV | |
| | Nu− | NCR nu−/nu−: | NCR nu−/nu−: | GFP+/nu−: | GFP−/nu−: furry, |
| | | nude mice | nude mice | furry, dark | dark eyes, does |
| | | with red eyes | with red eyes | eyes, glows | not glow green |
| | | | | green under UV | |

Pups from endogenous follicles would be nude, lacking fur, whereas pups from transplanted GFP follicles can have a fur with a 50% chance of glowing green (GFP/nu−). Breeding resulted in consecutive litters containing GFP+/nu− pups from one of the follicle recipient mice (Table IV).

TABLE IV
Breeding Cycle

| Chemotherapy | Animal | Cycle #1 | | | Cycle #2 | | | Cycle #3 | | | Total | Total |
|---|---|---|---|---|---|---|---|---|---|---|---|---|
| Dose | ID | Live | Dead | GFP | Live | Dead | GFP | Live | Dead | GFP | Pups | GFP |
| 12-100 | 6194 | 4 | 0 | 0 | 0 | 0 | 0 | 0 | 0 | 0 | 4 | 0 |
| | 6195 | 2 | 4 | 0 | 3 | 0 | 0 | 5 | 0 | 1 | 14 | 1 |
| | 6196 | 2 | 0 | 0 | 1 | 4 | 0 | 0 | 0 | 0 | 7 | 0 |
| | 6197 | 0 | 0 | 0 | 3 | 0 | 0 | 6 | 0 | 0 | 9 | 0 |
| | 6198 | 0 | 0 | 0 | 0 | 0 | 0 | 0 | 0 | 0 | 0 | 0 |
| 12-200 | 6242 | 6 | 0 | 0 | 0 | 0 | 0 | 0 | 0 | 0 | 6 | 0 |
| | 6243 | 7 | 0 | 0 | 0 | 0 | 0 | 0 | 0 | 0 | 7 | 0 |
| | 6245 | 0 | 0 | 0 | 0 | 0 | 0 | 0 | 0 | 0 | 0 | 0 |
| | 6246 | 0 | 0 | 0 | 4 | 0 | 0 | 0 | 0 | 0 | 4 | 0 |
| 24-100 | 6237 | 0 | 0 | 0 | 0 | 0 | 0 | 0 | 0 | 0 | 0 | 0 |
| | 6238 | 3 | 0 | 1 | 1 | 0 | 1 | 0 | 0 | 0 | 4 | 2 |
| | 6240 | 11 | 0 | 0 | 5 | 0 | 0 | 0 | 0 | 0 | 16 | 0 |
| | 6241 | 0 | 0 | 0 | 0 | 0 | 0 | 0 | 0 | 0 | 0 | 0 |

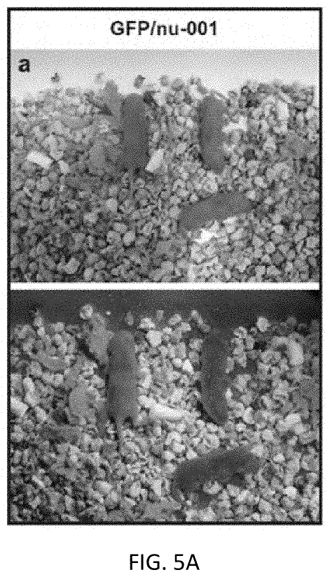

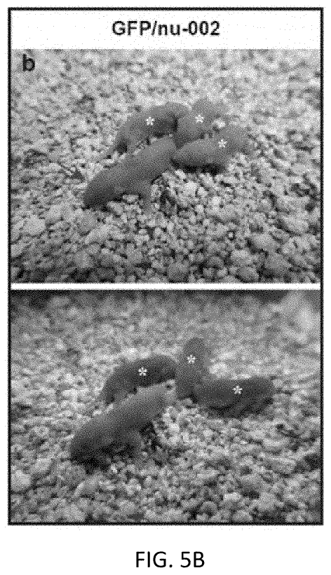

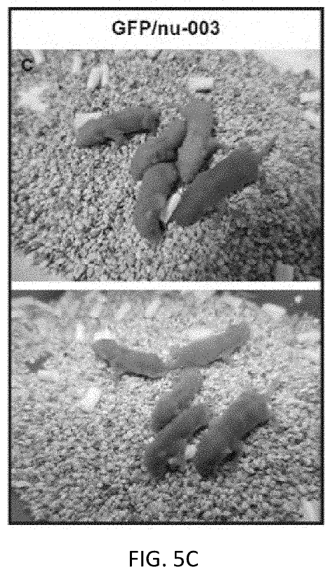

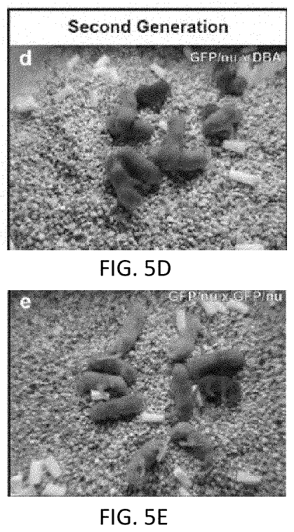

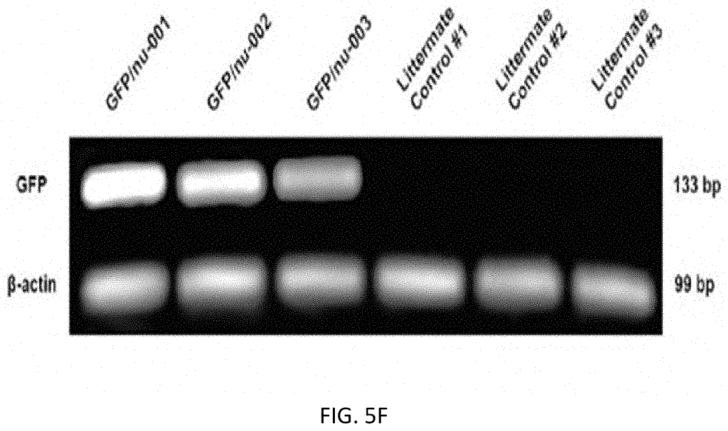

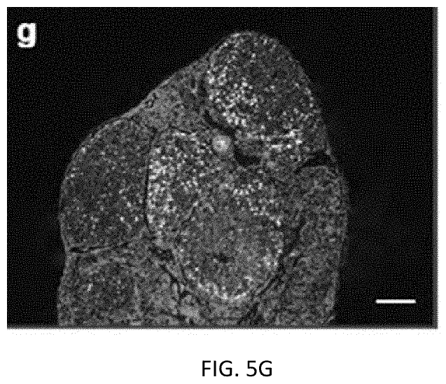

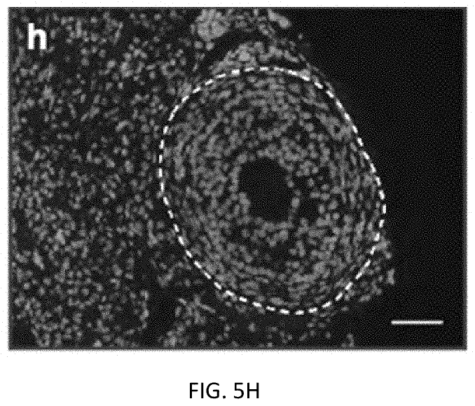

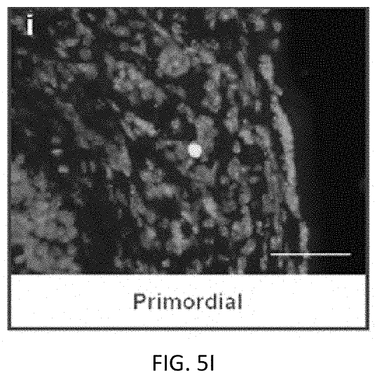

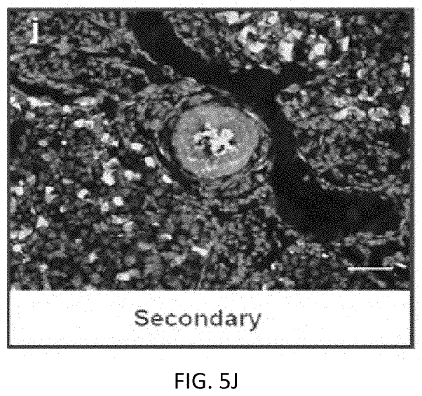

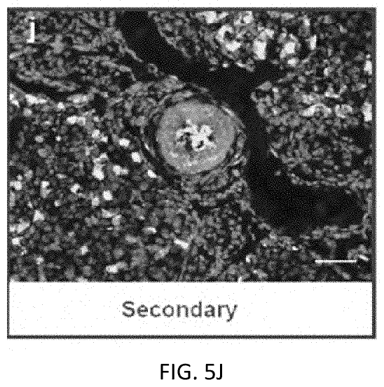

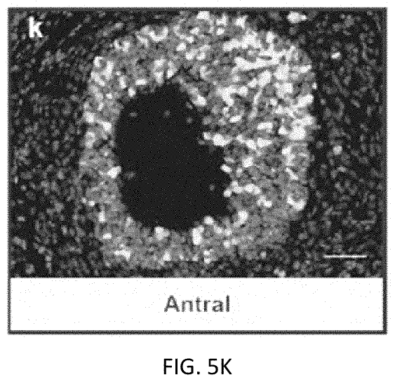

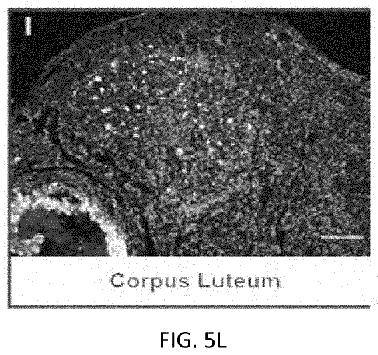

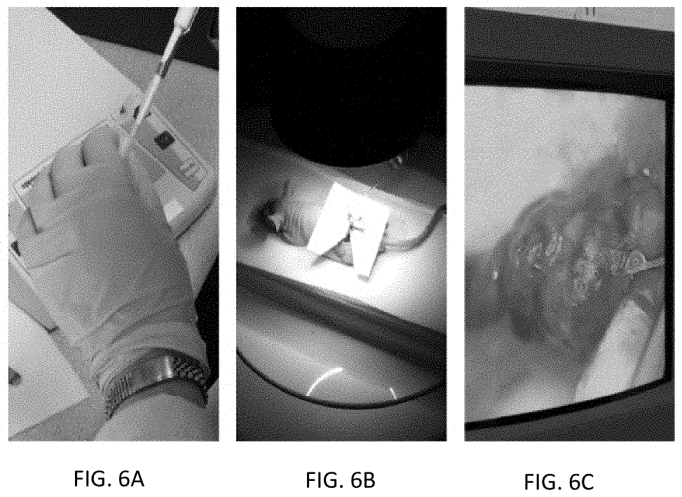

The first breeding cycle yielded one GFP pup (GFP/nu−001) out of three healthy offspring followed by a single GFP pup (GFP/nu−002) during the second cycle (FIGS. 5A and 5B). As a note, when litters were small, the pups were fostered into nude litters (as shown by the additional nude pups in FIG. 5B). Two GFP pups (GFP/nu−001 and GFP/nu−002) were born in consecutive liters from the same mother as a direct result of the follicle transplantation. The other follicle transplant recipient did not produce any offspring throughout the mating period. Only one of the control mice produced multiple litters with 11 pups during the first cycle and 5 pups during the second cycle with no GFP expression. None of the mice produced any litters during the third breeding trial.

Additionally, nude female mice treated with less severe dosage of chemotherapy, 12-100 (n=2) and 12-200 (n=2) mg/kg, underwent the same intraovarian follicle transplantation procedure. Upon mating, these mice produced a single GFP pup (GFP/nu−003) from the 12-100 dosage group during the third mating cycle (FIG. 5C), which provides additional evidence for potential long-term follicle viability post-transplant (Table IV). Another GFP pup (GFP/nu−003) was derived from a ciPOF female mouse (12-100 mg/kg busulfan-cyclophosphamide) during the third mating cycle (>100 days post-transplant). This demonstrates that an ISO can support injected follicles in a dose-independent manner and have long-term viability post-injection. Multiple litters of second generation pups were derived from both. Non-injected, chemotherapy-treated control mice from these two groups (n=5) produced a total of 20 nude, non-GFP offspring. In contrast, chemotherapy-treated, follicle-transplanted mice (n=4) led to the birth of 31 pups, with one mouse expressing GFP. A follow-up mating study was performed to test the reproductive health of the GFP pups generated from follicle transplantation. The GFP offspring resulting from follicle transplant (DBA-GFP+/nu−) were bred with DBA wild-type (GFP−/nu+) (Table V) and inbred with each mating pair (Table VI) producing multiple large litters of hemizygous (GFP+/−) and homozygous (GFP+/+) genetic backgrounds (FIG. 5D and Table VI). In each of the litters, GFP expression was clearly observed in the presence of a UV lamp and confirmed with genotyping (FIG. 5F).

TABLE IV
GFP/nu pups bred with DBA wild-type (GFP−/nu+) mic

| | GFP+/Nu+ | GFP+/Nu− | GFP−/Nu+ | GFP−/Nu− |
|---|---|---|---|---|
| GFP−/Nu+ | GFP+/− | GFP+/− | GFP−/− | GFP−/− |
| | Nu+/+ | Nu+/− | Nu+/+ | Nu+/− |
| GFP−/Nu+ | GFP+/− | GFP+/− | GFP−/− | GFP−/− |
| | Nu+/+ | Nu+/− | Nu+/+ | Nu+/− |
| GFP−/Nu+ | GFP+/− | GFP+/− | GFP−/− | GFP−/− |
| | Nu+/+ | Nu+/− | Nu+/+ | Nu+/− |
| GFP−/Nu+ | GFP+/− | GFP+/− | GFP−/− | GFP−/− |
| | Nu+/+ | Nu+/− | Nu+/+ | Nu+/− |

TABLE V
GFP/nu pups bred with GFP/nu pups.

| | GFP+/Nu+ | GFP+/Nu− | GFP−/Nu+ | GFP−/Nu− |
|---|---|---|---|---|
| GFP+/+ | GFP+/+ | GFP+/+ | GFP+/− | GFP+/− |
| | Nu+/+ | Nu+/− | Nu+/+ | Nu+/− |
| GFP+/Nu− | GFP+/+ | GFP+/+ | GFP+/− | GFP+/− |
| | Nu+/− | Nu−/− | Nu+/− | Nu−/− |
| GFP−/Nu+ | GFP+/− | GFP+/− | GFP−/− | GFP−/− |
| | Nu+/+ | Nu+/− | Nu+/+ | Nu+/− |
| GFP−/Nu− | GFP+/− | GFP+/− | GFP−/− | GFP−/− |
| | Nu+/− | Nu−/− | Nu+/− | Nu−/− |

TABLE VI
Multiple breeding cycles with the follicle-transplant derived GFP mice.

| Animal ID | Breeding Partner ID | Cycle | Live | Dead | GFP |
|---|---|---|---|---|---|
| GFP/nu-001 (F) | DBA-1683 (M) | 1 | 10 | 0 | 5 |
| | GFP/nu-003 (M) | 2 | 8 | 1 | 6 |
| GFP/nu-002 (F) | GFP/nu-003 (M) | 1 | 10 | 0 | 9 |
| | DBA-1684 (M) | 2 | 0 | 0 | 0 |
| DBA/2-1644 (F) | B6D2-853 (M) | 1 | 11 | 0 | 0 |
| | GFP/nu-003 (M) | 2 | 8 | 0 | 6 |
| | GFP/nu-003 (M) | 3 | 7 | 0 | 3 |

| | | | |
|---|---|---|---|
| Total Second Generation Pups | 43 | 1 | 29 |

Outbred (GFP/nu×DBA) and inbred (GFP/nu×GFP/nu) mice indicate that intraovarian follicle transplantation did not disrupt reproductive development (FIGS. 5D and 5E). Genotyping of the GFP pups and littermate controls was confirmed using standard PCR and gel electrophoresis (FIG. SF). GFP bands only appeared within the GFP mouse samples and none within the littermate samples. β-actin was used as an internal control appearing in each of the samples tested. Gels were cropped and processed to highlight the bands of interest. Ovarian tissues were excised from ciPOF follicle recipient mice after three breeding cycles (106 days on average). Immunofluorescence staining was performed using DAPI (endogenous cells) and GFP (transplanted cells) to evaluate follicle survival.

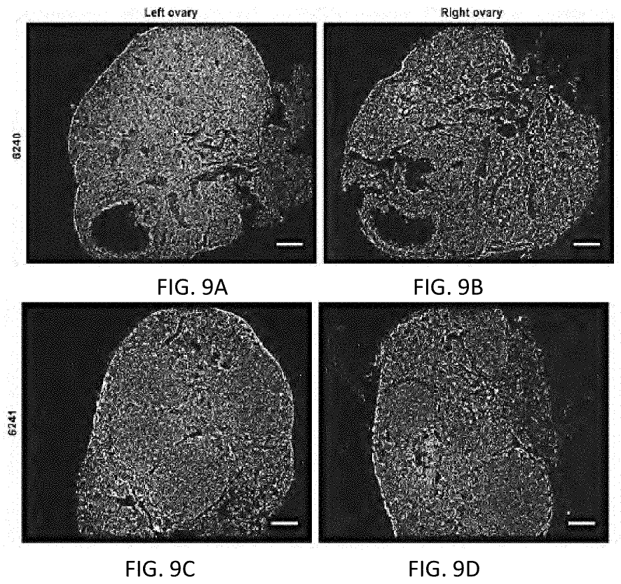

Intraovarian microinjection supports follicle longevity post-transplant: the effects of the disclosed techniques on follicle longevity post-transplant were evaluated. Immunofluorescent labeling of ovaries excised from ciPOF nude female mice after three breeding cycles (˜100 days) was performed. Comprehensive imaging demonstrated significant GFP expression throughout the transplanted tissues and suggests that multiple follicles remained viable post-transplantation (FIG. 5G). The GFP+ follicles within the transplanted ovary suggest that the transplanted cells integrated within the tissues and were actively developing. Transplanted ovaries also retained growing endogenous follicles, which may additionally be supported by the ISO components (FIG. 5H). The presence of an endogenous secondary follicle (GFP−) was confirmed (dotted line). Various stages of follicle development were also present among the transplanted tissues, including Non-injected control tissues that lacked GFP expression, and endogenous follicles appeared to be reduced in comparison to ovaries from transplanted mice (FIGS. 9A-9D). Merged DAPI and FITC immunofluorescence images of control ovaries (Animal ID: 6240 and 6241) show that non-injected control tissues have endogenous follicle growth, but appeared to have a reduced population of immature follicles in comparison to ovaries that received follicle transplant. No cells within the control tissues expressed GFP. However, definitive differences between residual endogenous follicles were not observed from the immunofluorescence images. Follicles at various stages of development including primordial (FIG. 5I), secondary (FIG. 5J), antral (FIG. 5K), and corpus luteum (FIG. 5L), which indicates that the ISO can facilitate long-term follicle integration, maintenance, and development, were identified.

Women who cryopreserve oocytes or embryos prior to gonadotoxic treatments can use assisted reproductive technologies (ART), such as IVF and embryo transfer to start a family. To preserve eggs or embryos, the patient must first undergo hormone stimulation to collect mature oocytes. Controlled ovarian stimulation requires two or more weeks and is not a viable option for patients who have not reached reproductive maturity or who cannot afford to postpone treatment. For these women, cryopreservation of intact ovarian tissues prior to treatment is the only potential option to naturally restore endocrine function and fertility. The current gold standard for fertility preservation in patients in remission is the autologous surgical transplantation of cryopreserved ovarian cortical strips. To date, there have been numerous successful procedures performed in humans, resulting in greater than 130 live-births; however, the efficiency of this method remains low, with live-birth rates ranging from 23-36% of patients achieving live-births. Although ovarian tissue transplantation has shown promise, it is an invasive procedure and carries a potential risk of reintroducing malignant cells back into the body. To address these concerns, several pre-clinical experimental approaches have been proposed, including in vitro follicle maturation (IVM), the development of an artificial ovary, and stem cell transplantation.

IVM consists of the isolation and culture of immature follicles to obtain meiotically-competent oocytes for IVF. IVM approaches have predominantly shifted from two-dimensional culture toward three-dimensional hydrogel-based follicle encapsulation, which has improved follicle morphology and intercellular signaling. The most commonly used hydrogel for IVM is alginate; although, there are several other options that have been examined including, fibrin, fibrin-alginate, and polyethylene glycol (PEG). Each of these materials provides a unique set of physical and biochemical properties, which allows them to support the growth and maturation of follicles in vitro. Successful application of IVM has been shown in mice leading to live-births; however, the pre-clinical translation of this approach for human follicles has been limited. Recently, follicle maturation has been attempted in vivo through the heterotopic subcutaneous transplant of a retrievable hydrogel seeded with immature follicles. Antral follicles developed in the hydrogel and germinal vesicle stage oocytes could be extracted, matured to MII stage, and fertilized, leading to the development of two and four-cell embryos. However, embryos were not transferred in this study, and pregnancies were not established.

As IVM has proven to be a major challenge for human follicles, several groups have pursued the development of an artificial ovary. This concept involves the isolation and sequestering of immature follicles in a bio-supportive scaffold that can be transplanted to recover ovarian function. Similar to IVM, various biomaterials are being examined as options to support the delivery, survival, and function of ovarian follicles in vivo. Recently, a fibrin gel supplemented with vascular endothelial growth factor (VEGF) was used to facilitate the transplantation of primordial follicles into the bursa of ovariectomized mice and gave rise to a healthy litter of pups. A 3D printed gelatin scaffold was used to examine the effects of pore geometry on follicle survival and achieved healthy pups through natural mating post-implantation in sterilized mice. While these methods have shown early success in an ovariectomy model of infertility, it is difficult to gauge the impact that chemotherapy would have on both implant integration and follicle survival. Moreover, these techniques require in vitro manipulation of follicles in preparation of the implants in addition to an invasive surgical transplantation procedure, which can have deleterious effects on reproductive outcomes.

The objective of the present study was to develop a minimally-invasive method to enhance intraovarian follicle microinjection with reduced in vitro manipulation and without the requirement of tissue culture. This was accomplished by solubilizing acellular ovarian scaffolds to create an injectable material that was able to reform following injection under physiological conditions. In addition, an adapted method was used for efficiently isolating follicles to reduce the total time ex vivo prior to transplantation. To mimic ciPOF experienced in the clinic, a single intraperitoneal injection of busulfan and cyclophosphamide was used to significantly reduce the endogenous follicle population in female recipient mice. The OECM hydrogel combined with freshly isolated GFP+ ovarian follicles were successfully delivered into the ovarian cortex, forming an ISO. Intraovarian follicle transplant aided by the OECM hydrogel gave rise to multiple, consecutive litters containing at least one pup expressing GFP.

The disclosed subject matter provides methods and devices for restoring fertility after chemotherapy. A major benefit of isolating follicles from the ovarian stroma is the potential to reduce malignant cells that can be introduced during transplantation. Follicle isolation can significantly decrease cancer cells prior to implantation. In order to increase the number of available donor follicles, isolated follicles were combined into a single batch and equally distributed for transplantation. The effects of this method on donor follicle survival after intraovarian microinjection were evaluated. As the number of injected follicles increases, it can also improve the potential fertility outcomes. In addition, the ratio of gel to follicles can also be a decisive factor in long-term follicle survival. For example, a higher follicle concentration can inhibit access to nutrients within the ISO triggering apoptosis or atresia.

OECM hydrogels, paired with intraovarian follicle microinjection, offer an enhanced cell delivery platform for fertility preservation. This therapeutic approach employs the innate remodeling capacity of the ECM to establish an ISO to support follicle transplants. The primary advantage of this strategy can provide a minimally invasive surgical intervention akin to transvaginal oocyte retrieval used for IVF. In addition, this approach has the potential to eliminate growth factor supplementation and reduce in vitro follicle manipulation. Overall, the restorative reproductive outcomes of the disclosed subject matter suggest that these alternative methods could be used to address current unmet clinical needs.

### 8.2 Example 2 Isolation and Delivery of Ovarian Follicles to Restore Reproductive Function for Women in Cancer Remission

Ovarian follicles are isolated from ovarian cortex after enzymatic treatment. Enzymatic isolation is performed by exposing a minced ovary to Liberase™ solution for two 5-minute incubations at 37° C. (200 RPM) with gentle pipetting to free the follicles from the connective tissues. Upon isolation, immature follicles (primordial, primary and secondary follicles) are selected and mixed with a 4 mg/ml of ovarian specific ECM hydrogel (pre-gel solution). The follicle/hydrogel mixture is kept on ice (˜4° C.) to prevent gelation and preserve follicle viability until transplant. Chemotherapy-treated patients are anesthetized, then surgically manipulated to expose the ovary. A microinjection needle is introduced directly into the ovarian cortex and a small volume (˜5-10 μL) of the follicle/hydrogel mixture is administered to the tissue. Gelation initiates upon exposure to physiologic conditions, which allows the follicles to be retained within the ovary.

After-injection, the patient is sutured to close the muscle and skin layers. Certain procedures such as transvaginal oocyte retrieval (TVOR) and amniocentesis, both performed under the guidance of ultrasound, can provide a minimally invasive blueprint for the delivery of the follicle/hydrogel mixture in human patients.

### 8.3 Example 3: Producing OECM Droplets

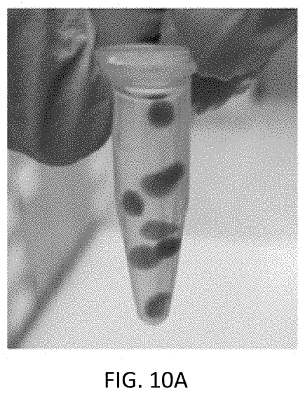

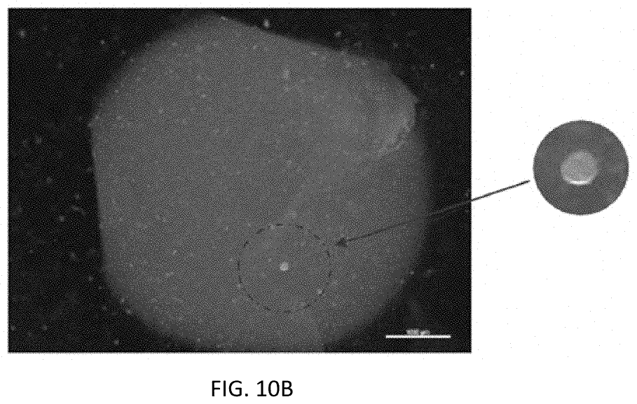

Pre-gel solution were prepared as described in Example 1. The pre-gel solution was kept at 4° C. 1XPBS or other desired encapsulation buffer was warmed to 37° C. A small volume (about 15-25 μL) of pre-gel volume was pipetted perpendicularly onto the surface of the warmed encapsulation buffer. Gel droplets were then allowed to completely form at 37° C. (approximately 10-20 minutes depending on ECM gelation kinetics). OECM droplets are shown in FIG. 10A. Follicles encapsulated in the OECM droplets were shown in FIG. 10B.

Although the presently disclosed subject matter and its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A method for restoring reproductive health and/or fertility in a subject in need thereof, comprising:
(a) providing an immature follicle;
(b) mixing the immature follicle with a hydrogel to form a follicle-hydrogel mixture; and
(c) delivering the follicle-hydrogel mixture to an ovary of the subject, wherein the immature follicle matures in vivo in the ovary.

2. The method of claim 1, wherein the subject has a cancer.

3. The method of claim 2, wherein the subject has previously received a cancer treatment.

4. The method of claim 1, wherein the immature follicle is isolated from an ovarian tissue of the subject.

5. The method of claim 1, wherein the immature follicle is isolated by an enzymatic treatment of the ovarian tissue.

6. The method of claim 1, wherein (b) further comprises storing the follicle-hydrogel mixture under conditions that prevent gelation and preserve a viability of the immature follicle.

7. The method of claim 1, wherein the immature follicle is selected from the group consisting of a primordial follicle, a primary follicle, a secondary follicle, and combinations thereof.

8. The method of claim 1, wherein (c) further comprises delivering the follicle-hydrogel mixture to the ovary of the subject by a microinjection needle.

9. The method of claim 1, wherein the hydrogel comprises an extracellular matrix of the decellularized ovarian tissue, wherein the extracellular matrix is present in a concentration between about 1 mg/ml to about 10 mg/ml.

10. The method of claim 1, wherein the hydrogel comprises one or more ovary-specific component.

11. A method for treating an ovarian-associated disease in a subject in need thereof, comprising:
(a) providing a therapeutic component;
(b) mixing the therapeutic component with an ovarian tissue derivative to form a therapeutic component-derivative mixture; and
(c) delivering the mixture to an ovary of the subject.

12. The method of claim 11, wherein the therapeutic component is selected from the group consisting of biomaterials, stem cells, therapeutic agents, and combinations thereof.

13. The method of claim 11, wherein the ovarian tissue derivative is selected from the group consisting of the ovarian tissue powder, ovarian tissue microparticles, ovarian tissue nanoparticles, ovarian tissue soluble fractions, ovarian tissue hydrogels, ovarian tissue matrix-bound nanovesicles, and combinations thereof.

14. The method of claim 11, wherein the therapeutic component-derivative mixture comprises an extracellular matrix of the decellularized ovarian tissue, wherein the extracellular matrix is present in a concentration between about 1 mg/ml to about 10 mg/ml.

15. The method of claim 11, wherein the therapeutic component-derivative mixture comprises one or more ovary-specific component.

16. A kit, comprising: a therapeutic component and an ovarian tissue derivative.

17. The kit of claim 16, further comprising instructions, the instructions comprise:
(a) mixing the therapeutic component with the ovarian tissue derivative to form a therapeutic component-derivative mixture; and
(b) delivering the mixture to an ovary of a subject.

18. The kit of claim 16, wherein the therapeutic component is selected from the group consisting of biomaterials, stem cells, therapeutic agents, and combinations thereof.

19. The kit of claim 16, wherein the ovarian tissue derivative comprises a decellularized ovarian tissue.

20. The kit of claim 16, wherein the ovarian tissue derivative is selected from the group consisting of the ovarian tissue powder, ovarian tissue microparticles, ovarian tissue nanoparticles, ovarian tissue soluble fractions, ovarian tissue hydrogels, ovarian tissue matrix-bound nanovesicles, and combinations thereof.
